SEQUENCE LISTING (5 sequences)

SEQ ID NO: 1 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 2 - protein, 609 aa, Homo sapiens [sequence omitted]

SEQ ID NO: 3 - protein, 896 aa, synthetic construct
LEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - protein, 1096 aa, synthetic construct
LEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 1107 aa, synthetic construct
LEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

(19) United States
(12) Patent Application Publication
(10) Pub. No.: US 2025/0123284 A1
(43) Pub. Date: Apr. 17, 2025

(54) DIAGNOSTIC METHODS FOR CANCER USING EPHRINB2 EXPRESSION

(71) Applicant: Vasgene Therapeutics Inc, Los Angeles, CA (US)

(72) Inventors: Jon Cogan, Azusa, CA (US); Valery Krasnoperov, Pasadena, CA (US)

(21) Appl. No.: 18/689,553

(22) PCT Filed: Sep. 7, 2022
(86) PCT No.: PCT/US2022/042812

§ 371 Date: Mar. 6, 2024

Related U.S. Application Data
(60) Provisional application No. 63/241,448, filed on Sep. 7, 2021.

(51) Int. Cl. G01N 33/574; G01N 33/68

(52) U.S. Cl. CPC G01N 33/57492; G01N 33/6872

(57) ABSTRACT

The present invention describes the use of EphrinB2 expression as a biomarker to evaluate the efficacy of treatment and to assist physicians in deciding on the course of a treatment in an individual suffering from a platinum resistant metastatic cancer. In one aspect, the invention provides methods to diagnose and select a subject with cancer for treatment using an EphB4-EphrinB2 inhibitor, or an EphB4-EphrinB2 inhibitor in combination with an immune stimulating drug, as front-line therapy for treatment of cancers. In another aspect, the invention provides methods to diagnose and select a subject with cancer for treatment using an EphB4-EphrinB2 inhibitor, or an EphB4-EphrinB2 inhibitor in combination with an immune stimulating drug, for treatment of a number of cancers where standard therapies have been shown to be ineffective, result in relapse, or are not even considered for use due to the type of cancer and related tumors. In another aspect, the invention provides methods to diagnose and select a subject with cancer for treatment using an EphB4-EphrinB2 inhibitor, or an EphB4-EphrinB2 inhibitor in combination with an immune stimulating drug, for treatment of a number of cancers wherein the subject is currently on a course of treatment with an immune stimulating drug.

This application claims benefit of U.S. Provisional Application No. 63/241,448, filed on Sep. 7, 2021, incorporated in its entirety by reference herein.

### SEQUENCE LISTING

The instant application contains a Sequence Listing in the form of a “paper copy” (PDF File) and a file containing the referenced sequences (SEQ ID NOs: 1-5) in computer readable form (ST25 format text file) which is submitted herein. The Sequence Listing is shown using standard three letter code for amino acids, as defined in 37 C.F.R. 1.822.

### TECHNICAL FIELD

Today, cancer remains a major cause of death worldwide despite the numerous advanced diagnostic and therapeutic methods that have been developed. In humans, cancers become established after a primary genetic event by a number of mechanisms that include, but are not limited to, increased cellular metabolism and growth rate, stimulation of angiogenesis thereby increasing blood supply to the tumor, and disregulation of signaling pathways and tumor suppressors. Curative treatment protocols in clinical oncology remain reliant upon a combination of surgical resection, ionizing radiation, and cytotoxic chemotherapy. The major barrier to successful treatment and prevention of cancer lies in the fact that many cancers still fail to respond to the current chemotherapeutic and immunotherapy intervention, and many individuals suffer a recurrence or death, even after aggressive therapy. Moreover, tumors may become resistant to anti-cancer drugs by a number of mechanisms that include, but are not limited to, expulsion of the drug from the cell, occurrence of mutations that prevent binding of the drug to its target, and occurrence of additional mutations in genes and their protein products unrelated to the drug target. To address these shortcomings, there has been a trend in drug discovery to develop targeted therapies capable of modulating signaling axes dysregulated in cancers. A key feature of targeted therapeutic approaches is reliable diagnostic and prognostic biomarkers. There are now many FDA approved antibodies and small molecules that allow for therapeutic manipulation of a myriad of clinically relevant targets.

Immunotherapy using agonistic, antagonistic, or blocking antibodies to co-stimulatory or co-inhibitory molecules (immune checkpoints) has been an area of extensive research and clinical evaluation. Immune checkpoint proteins include CTLA-4, PD-1, PD-L1, LAG-3, and TIM-3 as well as several others (Sharpe et al., Nat Immunol, 8:239-45, 2007). Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll D M., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), and OX-40, CD137, GITR, LAG3, TIM-3, and VISTA (see, e.g., Sharon et al., Chin J Cancer., 33(9): 434-444, September 2014; Hodi et al., N Engl J Med, 2010; Topalian et al., N Engl J Med, 366:2443-54) are being evaluated as new, alternative immunotherapies to treat patients with proliferative diseases such as cancer, and in particular, patients with refractory and/or recurrent cancers. Despite significant advances in diagnosis and therapy, cancer remains a major common cause of morbidity and mortality.

Currently, checkpoint inhibitor therapy has become the preferred first, second, or third-line therapy for many cancers, and PD1/PDL1 antibodies have changed the treatment paradigm for several cancers. Despite these significant advancements, there remains a major need to improve on the current state-of-the-art. For example, checkpoint inhibitor therapy remains limited by concerns over potential severe side effects and the fact that many tumors lack the targeted antigen and will therefore evade treatment. In general, about 20% of patients with various cancers respond to PD-1/PD-L1 antibodies or CTLA-4 antibodies and overall survival remains less than a year in most instances and objective response rates are modest, underscoring the large unmet need for nearly 70-80% of these patients.

Eph (Erythropoietin Producing Hepatoma) receptor and ligand are part of the largest family of receptor tyrosine kinases (RTKs). The family is subdivided into class A and class B, based on sequence homology and binding affinity for two distinct types of membrane-anchored ephrin ligands. Each Eph receptor and ligand can bind to multiple ligands and receptors and certain receptors have been postulated as putative tumor suppressors and others as tumor promoters (Vaught et al, Breast Cancer Res, 10(6):217-224, 2008). EphrinB2 and its high affinity cognate receptor, EphB4, are transmembrane proteins that are induced in tumor vessels and regulate immune cell trafficking. Inhibition of the EphB4-EphrinB2 interaction has a direct inhibitory effect on tumor cell proliferation in vitro and ex-vivo. Polypeptide agents that inhibit EphB4 or EphrinB2 mediated functions have been previously described by the present inventors (see, e.g., U.S. Pat. Nos. 7,381,410; 7,862,816; 7,977,463; 8,063,183; 8,273,858; 8,975,377; 8,981,062; 9,533,026; each hereby incorporated by reference in their entirety for all purposes).

The present inventors have determined that soluble extracellular fragment of EphB4 fused to albumin (sEphB4-HSA) blocks interaction between Ephrin-B2 and EphB4, and blocks bidirectional signaling, thus promoting immune cell trafficking, and inducing an anti-tumor immune response in various cancers. As such, the present inventors are developing an EphB4-EphrinB2 inhibitor, “sEphB4-HSA” (soluble extracellular fragment of EphB4 tyrosine kinase receptor fused to Human Serum Albumin) for the treatment of various cancers. sEphB4-HSA consists of the extracellular domain of human EphB4 receptor (sEphB4) fused in frame with human serum albumin (HSA). This fusion with HSA enhances the pharmacokinetics of sEphB4. sEphB4-HSA binds to the ligand of EphB4 tyrosine kinase receptor: the transmembrane protein Ephrin-B2. Through this binding, it blocks endogenous EphB tyrosine kinase receptors from interacting with EphrinB2. Evidence generated to date indicates that sEphB4-HSA reduces angiogenesis in tumors—thus starving tumors of blood and inhibits EphrinB2's ability to suppress recruitment of T cells to tumors—thus increasing T cell recruitment. EphB4 is a survival factor in several tumor types such as squamous cell carcinoma, urothelial carcinoma, colon cancer, lung cancer, mesothelioma, ovarian cancer, pancreatic cancer, prostate cancer and others. EphrinB2 is a survival factor in some tumors such as Kaposi's sarcoma. Blocking bidirectional signaling blocks activation of EphB4 and thus forward signaling leading to cell stasis or cell death.

The present invention is directed, in part, to the use of EphrinB2 expression as a biomarker to evaluate the efficacy of treatment and to assist physicians in deciding on the course of a treatment in an individual suffering from a metastatic cancer.

### INCORPORATION BY REFERENCE

Patent documents U.S. Pat. Nos. 7,381,410; 7,862,816; 7,977,463; 8,063,183; 8,273,858; 8,975,377; 8,981,062; 9,533,026; PCT/US2020/018160; PCT/US2020/023215 and all references disclosed herein are hereby incorporated by reference in their entirety for all purposes.

### DISCLOSURE OF THE INVENTION

The present invention is based in part on the surprising discoveries that: 1) EphrinB2 expression appears to correlate with response to EphrinB2 targeted therapy as a single agent and/or in combination therapy using a polypeptide agent that inhibits EphB4 or EphrinB2 mediated functions (“EphB4-EphrinB2 inhibitors”)(e.g., sEphB4-HSA) in combination with an immune stimulating drug (including but not limited to antagonistic antibodies to PD-1/PD-L1, CTLA-4, LAG3, TIM3, TIGIT, OX40 ligand) as front-line therapy in locally advanced or metastatic urothelial/bladder cancer; and 2) higher EphrinB2 expression appears to correlate with a lower response to monotherapy using an immune stimulating drug (e.g., a PD-1/PD-L1 antagonistic antibody) in a metastatic urothelium carcinoma post systemic chemotherapy study.

Accordingly, the present invention describes the use of EphrinB2 expression as a biomarker to evaluate the efficacy of treatment and to assist physicians in deciding on the course of a treatment in an individual suffering from a metastatic cancer. In various aspects, the invention provides methods to diagnose and select a subject with cancer for treatment using an EphB4-EphrinB2 inhibitor, or an EphB4-EphrinB2 inhibitor in combination with an immune stimulating drug, as front-line therapy for treatment of cancers. In various aspects, the invention provides methods to diagnose and select a subject with cancer for treatment using an EphB4-EphrinB2 inhibitor, or an EphB4-EphrinB2 inhibitor in combination with an immune stimulating drug, for treatment of a number of cancers where standard therapies have been shown to be ineffective, result in relapse, or are not even considered for use due to the type of cancer and related tumors. In various aspects, the invention provides methods to diagnose and select a subject with cancer for treatment using an EphB4-EphrinB2 inhibitor, or an EphB4-EphrinB2 inhibitor in combination with an immune stimulating drug, for treatment of a number of cancers wherein the subject is currently on a course of treatment with an immune stimulating drug. In some embodiments, the individual suffers from a platinum resistant metastatic cancer. In some embodiments, the individual has not received prior platinum chemotherapy, or is too unhealthy to receive platinum chemotherapy.

In some embodiments, the method of diagnosing and selecting a subject with cancer for treatment using an EphB4-EphrinB2 inhibitor in combination with an immune stimulating drug comprises: i) detecting the level of EphrinB2 expression in a biological sample from a subject who has been diagnosed with a cancer; ii) selecting the subject for treatment using an EphB4-EphrinB2 inhibitor in combination with an immune stimulating drug as front-line therapy when EphrinB2 expression is 1% or greater.

In some embodiments, the method of diagnosing and selecting a subject with cancer for treatment using an EphB4-EphrinB2 inhibitor comprises: i) detecting the level of EphrinB2 expression in a biological sample from a subject who has been diagnosed with a cancer; ii) selecting the subject for treatment using an EphB4-EphrinB2 inhibitor as front-line therapy when EphrinB2 expression is 1% or greater.

In some embodiments, the method of diagnosing a subject with cancer comprises: i) detecting the level of EphrinB2 expression in a biological sample from a subject who has been diagnosed with a cancer and is currently on a course of treatment with an immune stimulating drug; and ii) changing therapy when EphrinB2 expression is 1% or greater.

In some embodiments, the polypeptide agent that inhibits EphB4 or EphrinB2 mediated functions is a monomeric ligand binding portion of the EphB4 protein or EphrinB2 protein, or an antibody that binds to and affects EphB4 or EphrinB2. In some embodiments, the polypeptide agent is a soluble EphB4 (sEphB4) polypeptide that binds specifically to an EphrinB2 polypeptide and comprises an amino acid sequence of an extracellular domain of an EphB4 protein. In some embodiments, the sEphB4 polypeptide comprises a globular domain of an EphB4 protein. In some embodiments, the agent that inhibits EphB4 or EphrinB2 mediated functions is a nucleic acid therapeutic agent. In some embodiments, the nucleic acid therapeutic agent that inhibits EphB4 or EphrinB2 mediated functions is oligonucleotide DNA or siRNA which targets EphrinB2 or EphB4.

In some embodiments, the sEphB4 polypeptide comprises a sequence selected from the group consisting of a sequence that is at least 90% identical to residues 1-522, at least 90% identical to residues 1-412, and at least 90% identical to residues 1-311 of the amino acid sequence of SEQ ID NO: 1. In some embodiments, the sEphB4 polypeptide may comprise a sequence encompassing the globular (G) domain (amino acids 29-197 of SEQ ID NO; 1), and optionally additional domains, such as the cysteine-rich domain (amino acids 239-321 of SEQ ID NO: 1), the first fibronectin type 3 domain (amino acids 324-429 of SEQ ID NO: 1) and the second fibronectin type 3 domain (amino acids 434-526 of SEQ ID NO: 1). In some embodiments, the sEphB4 polypeptide will comprise amino acids 1-537 of SEQ ID NO: 1. In some embodiments, the sEphB4 polypeptide will comprise amino acids 1-427 of SEQ ID NO: 1. In some embodiments, the sEphB4 polypeptide will comprise amino acids 1-326 of SEQ ID NO: 1. In some embodiments, the sEphB4 polypeptide will comprise amino acids 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537 of SEQ ID NO: 1. In some embodiments, the sEphB4 polypeptide will comprise amino acids 16-197, 16-312, 16-321, 16-326, 16-412, 16-427, 16-429, 16-526, and 16-537 of SEQ ID NO: 1.

In some embodiments, a sEphB4 polypeptide may be prepared in a multimeric form, by, for example, expressing as an Fc fusion protein or fusion with another multimerization domain.

In some embodiments, the sEphB4 polypeptide will further comprise an additional component that confers increased serum half-life while still retaining EphrinB2 binding activity. In some embodiments, the sEphB4 polypeptides are monomeric and are covalently linked to one or more polyoxyaklylene groups (e.g., polyethylene, polypropylene). In some embodiments, the sEphB4 polypeptide is covalently linked to a polyethylene glycol (PEG) group(s) (hereinafter “sEphB4-PEG”).

In some embodiments, the sEphB4 polypeptide is stably associated with a second stabilizing polypeptide that confers improved half-life without substantially diminishing EphrinB2 binding. In some embodiments, the stabilizing polypeptide is immunocompatible with human patients (or animal patients, where veterinary uses are contemplated) and will have little or no significant biological activity. In some embodiments, the sEphB4 polypeptide is associated covalently or non-covalently with an albumin selected from the group consisting of a human serum albumin (HSA) (hereinafter “sEphB4-HSA”) and bovine serum albumin (BSA) (hereinafter “sEphB4-BSA”). In some embodiments, the sEphB4-HSA comprises residues 16-197 of SEQ ID NO: 1 directly fused to residues 25-609 of SEQ ID NO: 2. In some embodiments, the sEphB4-HSA comprises residues 16-312 of SEQ ID NO: 1 directly fused to residues 25-609 of SEQ ID NO: 2. In some embodiments, the sEphB4-HSA comprises residues 16-321 of SEQ ID NO: 1 directly fused to residues 25-609 of SEQ ID NO: 2. In some embodiments, the sEphB4-HSA comprises residues 16-326 of SEQ ID NO: 1 directly fused to residues 25-609 of SEQ ID NO: 2. In some embodiments, the sEphB4-HSA comprises residues 16-412 of SEQ ID NO: 1 directly fused to residues 25-609 of SEQ ID NO: 2. In some embodiments, the sEphB4-HSA comprises residues 16-427 of SEQ ID NO: 1 directly fused to residues 25-609 of SEQ ID NO: 2. In some embodiments, the sEphB4-HSA comprises residues 16-429 of SEQ ID NO: 1 directly fused to residues 25-609 of SEQ ID NO: 2. In some embodiments, the sEphB4-HSA comprises residues 16-526 of SEQ ID NO: 1 directly fused to residues 25-609 of SEQ ID NO: 2. In some embodiments, the sEphB4-HSA comprises residues 16-537 of SEQ ID NO: 1 directly fused to residues 25-609 of SEQ ID NO: 2. In some embodiments, the sEphB4-HSA comprises the amino acid sequence set forth in SEQ ID NO: 3. In some embodiments, the sEphB4-HSA comprises the amino acid sequence set forth in SEQ ID NO: 4. In some embodiments, the sEphB4-HSA comprises the amino acid sequence set forth in SEQ ID NO: 5.

In some embodiments, a sEphB4 polypeptide may be prepared in a multimeric form, by, for example, expressing as a fusion protein with a molecule that blocks signaling or blocks EphrinB2 interaction with Eph receptors, including but not limited to antisense oligonucleotides, siRNA, and gene editing like CRISPR/CAS.

In some embodiments, the immune stimulating drug is an antagonist to an immune-checkpoint protein antigen selected from the group consisting of: SIRP (expressed on macrophage, monocytes, dendritic cells), CD47 (highly expressed on tumor cells and other cell types), TIGIT (immune receptor present on some T cells and natural killer cells), VISTA (expressed on monocytes, dendritic cells, B cells, T cells), CD152 (expressed by activated CD8+ T cells, CD4+ T cells and regulatory T cells), CD279 (expressed on tumor infiltrating lymphocytes, expressed by activated T cells (both CD4 and CD8), regulatory T cells, activated B cells, activated NK cells, anergic T cells, monocytes, dendritic cells), CD274 (expressed on T cells, B cells, dendritic cells, macrophages, vascular endothelial cells, pancreatic islet cells), and CD223 (expressed by activated T cells, regulatory T cells, anergic T cells, NK cells, NKT cells, and plasmacytoid dendritic cells). In some embodiments, the immune stimulating drug is selected from the group consisting of an anti-PD-1 Ab, anti-PD-L1 Ab, anti-CTLA Ab, anti-TIGIT Ab, anti-LAG3 antibody, anti-TIM3 antibody, and combinations thereof.

In some embodiments, EphrinB2 expression is determined by protein expression using a method selected from the group consisting of immunohistochemistry (IHC), immunofluorescence, flow cytometry, and Western blot. In some embodiments, the mRNA expression level is determined using a method selected from the group consisting of quantitative polymerase chain reaction (qPCR), reverse transcription qPCR (RT-qPCR), RNA sequencing, microarray analysis, in situ hybridization, and serial analysis of gene expression (SAGE).

In some embodiments, the biological sample is selected from the group consisting of a tissue sample, a blood sample, a serum sample, a plasma sample, a cerebrospinal fluid (CSF) sample, an ascites fluid sample, and a cell culture sample.

In some embodiments, the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias. In some embodiments, the cancer is ovarian cancer. In some embodiments, the cancer is a breast cancer. In some embodiments, the cancer is a RAS (e.g., KRAS, HRAS, NRAS) mutant cancer. In some embodiments, the cancer is a cancer with PTEN loss.

In some embodiments, the subject previously responded to treatment with an anti-cancer therapy, but, upon cessation of therapy, suffered relapse (hereinafter “a recurrent cancer”). In some embodiments, the subject has resistant or refractory cancer. In some embodiments, the cancer is refractory to platinum-based chemotherapy. In some embodiments, the cancer is refractory to immunotherapy treatment. In some embodiments, the cancer is refractory to treatment with a chemotherapeutic agent. In some embodiments, the cancer is refractory to treatment using depleting antibodies to specific tumor antigens. In some embodiments, the cancer is refractory to treatment using agonistic, antagonistic, or blocking antibodies to co-stimulatory or co-inhibitory molecules (immune checkpoints). In some embodiments, the cancer is refractory to targeted treatment with an immunoconjugate, antibody-drug conjugate (ADC), or fusion molecule comprising a depleting antibody to specific tumor antigens tumor antigen and a cytotoxic agent. In some embodiments, the cancer is refractory to targeted treatment with a small molecule kinase inhibitor. In some embodiments, the cancer is refractory to treatment using surgery. In some embodiments, the cancer is refractory to treatment using stem cell transplantation. In some embodiments, the cancer is refractory to treatment using radiation. In some embodiments, the cancer is refractory to combination therapy involving, for example, two or more of: immunotherapy treatment, treatment with a platinum based chemotherapeutic agent, treatment with a tumor antigen-specific, depleting antibody, treatment with a immunoconjugate, ADC, or fusion molecule comprising a tumor antigen-specific, depleting antibody and a cytotoxic agent, targeted treatment with a small molecule kinase inhibitor, treatment using surgery, treatment using stem cell transplantation, and treatment using radiation.

In some embodiments, the method for treating a cancer in a subject further comprises a second therapy selected from the group consisting of: small molecule kinase inhibitor targeted therapy, surgery, cytoreductive therapy, cytotoxic chemotherapy, and immunotherapy. In some embodiments, the combination therapy will be synergistic. In some embodiments, the second therapy is cytoreductive therapy and the combination may increase the therapeutic index of the cytoreductive therapy. In some embodiments, the cytoreductive therapy may act in a DNA repair pathway. In some embodiments, the cytoreductive therapy is radiation therapy. In some embodiments, the combination may be synergistic.

In some embodiments, the second therapy is a chemotherapeutic agent is selected from the group consisting of: daunorubicin, adriamycin (doxorubicin), epirubicin, idarubicin, anamycin, MEN 10755, etoposide, teniposide, vinblastine, vincristine, vinorelbine (NAVELBINE); vindesine, vindoline, vincamine, mechlorethamine, cyclophosphamide, melphalan (L-sarcolysin), carmustine (BCNU), lomustine (CCNU), semustine (methyl-CCNU), streptozocin, chlorozotocin, cytarabine (CYTOSAR-U), cytosine arabinoside, fluorouracil (5-FU), floxuridine (FUdR), thioguanine (6-thioguanine), mercaptopurine (6-MP), pentostatin, fluorouracil (5-FU), methotrexate, 10-propargyl-5,8-dideazafolate (PDDF, CB3717), 5,8-dideazatetrahydrofolic acid (DDATHF), leucovorin, cisplatin (cis-DDP), carboplatin, oxaliplatin, hydroxyurea, gemcitabine, and N-methylhydrazine.

In some embodiments, the second therapy will comprise administration of a poly (ADP-ribose) polymerase inhibitor (PARP) inhibitor. In some embodiments, the PARP inhibitor is selected from the group consisting of ABT-767, AZD 2461, BGB-290, BGP 15, CEP 9722, E7016, E7449, fluzoparib, INO1001, JPI 289, MP 124, niraparib, olaparib, ON02231, rucaparib, SC 101914, talazoparib, veliparib, WW 46, or salts or derivatives thereof. olaparib, rucaparib, niraparib, talazoparib and veliparib. In some embodiments, the combination may be synergistic.

In some embodiments, the method of treatment will comprise the administration of sEphB4-HSA in combination with pegylated liposomal doxorubicin (PLD). In some embodiments, the method of treatment will comprise the administration of sEphB4-HSA in combination with paclitaxel. In some embodiments, the combination may be synergistic.

In some embodiments, the second therapy will comprise immunotherapy selected from, but not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAG3, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-□□□IFN-3 and IFN-γ; treatment using therapeutic vaccines such as sipuleucel-T; treatment using dendritic cell vaccines, or tumor antigen peptide vaccines; treatment using oncolytic virus therapy (T-VEC); treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; wherein the combination therapy provides increased effector cell killing of tumor cells, i.e., a synergy exists between sEphB4-HSA and the immunotherapy when co-administered.

In some embodiments, the additional therapy comprises administration of an antibody that specifically binds an immune-checkpoint protein antigen from the list including, but not limited to, CD276, CD272, CD152, CD223, CD279, CD274, TIM-3 and B7-H4; or any immune-checkpoint protein antigen antibody taught in the art. In some embodiments, the PD-1 inhibitor is selected from the group consisting of, but not limited to, nivolumab (Bristol-Myers Squibb)(Drugbank 09035; Drugbank 06132), pembrolizumab (Merck)(Drugbank 09037) and pidilizumab (Medivation)(Drugbank 15383). In some embodiments, the CTLA-4 inhibitor is selected from the group consisting of, but not limited to, ipilimumab (Bristol-Myers Squibb)(Drugbank 06186) and tremelimumab (MedImmune)(Drugbank 11771).

### BRIEF DESCRIPTION OF THE DRAWINGS

FIG. 1 provides pictures of EphrinB2 Immuno-histochemistry (IHC) and EphrinB2 In situ hybridization (ISH/RNAScope) of EphrinB2 positive non-responders.

FIG. 2 provides pictures of EphrinB2 Immuno-histochemistry (IHC) and EphrinB2 In situ hybridization (ISH/RNAScope) of 5 responders.

FIG. 3 provides pictures of EphrinB2 Immuno-histochemistry (IHC) and EphrinB2 In situ hybridization (ISH/RNAScope) of cell pellet controls of various isogenic CHO cell lines: wild type or ectopically expressing human EphrinB2, and 2 urothelial cancer tissues.

### MODE(S) FOR CARRYING OUT THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, the cells of interest for detection, analysis, classification, or treatment in the present application include precancerous (e.g., benign), malignant, pre-metastatic, and non-metastatic cells.

The term “primary tumor” refers to all neoplastic cell growth and proliferation, whether malignant or benign, and all pre-cancerous and cancerous cells and tissues located at the anatomical site where the autonomous, unregulated growth of the cells initiated, for example the organ of the original cancerous tumor. Primary tumors do not include metastases.

The “pathology” of cancer includes all phenomena that compromise the well-being of the patient. This includes, without limitation, abnormal or uncontrollable cell growth, primary tumor growth and formation, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor (e.g., the organ containing the primary tumor). Metastasis can also be defined as several steps of a process, such as the departure of cancer cells from an original tumor site (e.g., primary tumor site) and migration and/or invasion of cancer cells to other parts of the body.

Depending on the nature of the cancer, an appropriate patient sample is obtained. As used herein, the phrase “cancerous tissue sample” refers to any cells obtained from a cancerous tumor. In the case of solid tumors which have not metastasized (for example a primary tumor), a tissue sample from the surgically removed tumor will typically be obtained and prepared for testing by conventional techniques.

By “early stage cancer” or “early stage tumor” is meant a cancer that is not invasive or metastatic or is classified as a Stage 0, 1, or 2 cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include bladder cancer (e.g., urothelial bladder cancer (e.g., transitional cell or urothelial carcinoma, non-muscle invasive bladder cancer, muscle-invasive bladder cancer, and metastatic bladder cancer) and non-urothelial bladder cancer), squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, as well as head and neck cancer and hematological malignancies.

Tumors of interest for treatment with the methods of the invention include solid tumors, e.g., carcinomas, gliomas, melanomas, sarcomas, and the like. Ovarian cancer and breast cancer is of particular interest. Carcinomas include a variety of adenocarcinomas, for example in prostate, lung, etc.; adernocartical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (e.g., glioblastoma), lung, breast, skin, etc. Including in the designation of soft tissue tumors are neoplasias derived from fibroblasts, myofibroblasts, histiocytes, vascular cells/endothelial cells and nerve sheath cells. Tumors of connective tissue include sarcomas; histiocytomas; fibromas; skeletal chondrosarcoma; extraskeletal myxoid chondrosarcoma; clear cell sarcoma; fibrosarcomas, etc. Hematologic cancers include leukemias and lymphomas, e.g., cutaneous T cell lymphoma, acute myeloid leukemia (AML), chronic myeloid leukemia (CML), acute lymphoblastic leukemia (ALL), non-Hodgkins lymphoma (NHL), etc.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatmentor respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor cells. In some embodiments, the cancer is resistant to standard therapies. In some embodiments, the cancer is a chemoresistant cancer. In some embodiments, the cancer is a platinum resistant cancer.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

As used herein, when evaluating EphrinB2 expression as a biomarker, EphrinB2 expression is positive with 1% or more of the cells showing membrane signal.

As used herein, “treatment” is an approach for obtaining beneficial or desired clinical results. For purposes of this invention, beneficial or desired clinical results include, but are not limited to, any one or more of: alleviation of one or more symptoms; diminishment of extent of disease; preventing or delaying spread (e.g., metastasis, for example metastasis to the lung or to the lymph node) of disease; preventing or delaying recurrence of disease; stabilizing, delaying or slowing of disease progression; amelioration of the disease state; remission (whether partial or total); and improving quality of life. Also encompassed by “treatment” is a reduction of pathological consequence of a proliferative disease. The methods of the invention contemplate any one or more of these aspects of treatment.

Treating may refer to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction, elimination, or prevention of the disease, symptoms of the disease, or side effects of the disease in the subject.

The phrase “synergistic effect” refers to the effect achieved when the active ingredients used together is greater than the sum of the effects that results from using the active ingredients separately.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5 times, 2.0 times, 2.5 times, or 3.0 times the length of the treatment duration.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “objective response rate” (ORR) refers to the sum of complete response (CR) rate and partial response (PR) rate.

As used herein, “overall survival” (OS) refers to the percentage of individuals in a group who are likely to be alive after a particular duration of time.

As used herein, polypeptide agents that inhibit EphB4 or EphrinB2 mediated functions (“EphrinB2/EphB4 inhibitors”) are used to refer to inhibitory, activating, or modulating molecules, respectively, identified using in vitro and in vivo assays for receptor or ligand binding or signaling, e.g., ligands, receptors, agonists, antagonists, and their homologs and mimetics.

The EphrinB2/EphB4 inhibitors having the desired pharmacological activity may be administered in a physiologically acceptable carrier to a host to inhibit EphB4 or EphrinB2 mediated functions. The therapeutic agents may be administered in a variety of ways, orally, topically, parenterally e.g., intravenous, subcutaneously, intraperitoneally, by viral infection, intravascularly, etc. Intravenous delivery is of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer, including without limitation, adenocarcinoma of the ovary or prostate, breast cancer, glioblastoma, etc., including those who have undergone or are candidates for resection (surgery) to remove cancerous tissue. Subjects may be human, but also include other mammals, particularly those mammals useful as laboratory models for human disease, e.g., mouse, rat, etc.

The term “diagnosis” is used herein to refer to the identification of a molecular or pathological state, disease or condition, such as the identification of a virus infection.

A “therapeutically effective amount” refers to the amount of a compound that, when administered to a subject for treating breast or ovarian cancer, is sufficient to affect such treatment of the cancer. The “therapeutically effective amount” may vary depending, for example, on the sEphB4-HSA polypeptide or immune stimulating drug selected, the stage of the cancer, the age, weight and/or health of the patient and the judgment of the prescribing physician. An appropriate amount in any given instance may be readily ascertained by those skilled in the art or capable of determination by routine experimentation.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).).

In the case of a cancer or a tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

“In combination with”, “combination therapy” and “combination products” refer, in certain embodiments, to the concurrent administration to a patient of a first therapeutic and the compounds as used herein. In some embodiments, the combination products are administered non-concurrently. When administered in combination, each component can be administered at the same time or sequentially in any order at different points in time. Thus, each component can be administered separately but sufficiently closely in time so as to provide the desired therapeutic effect.

“Concomitant administration” of a known cancer therapeutic drug with a pharmaceutical composition of the present invention means administration of the drug and AXL variant at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

### EXEMPLARY EMBODIMENTS

The present invention describes the use of EphrinB2 expression as a biomarker to evaluate the efficacy of treatment and to assist physicians in deciding on the course of a treatment in an individual suffering from a metastatic cancer. In various aspects, the invention provides methods to diagnose and select a subject with cancer for treatment using an EphB4-EphrinB2 inhibitor, or an EphB4-EphrinB2 inhibitor in combination with an immune stimulating drug, as front-line therapy for treatment of cancers. In various aspects, the invention provides methods to diagnose and select a subject with cancer for treatment using an EphB4-EphrinB2 inhibitor, or an EphB4-EphrinB2 inhibitor in combination with an immune stimulating drug, for treatment of a number of cancers where standard therapies have been shown to be ineffective, result in relapse, or are not even considered for use due to the type of cancer and related tumors. In various aspects, the invention provides methods to diagnose and select a subject with cancer for treatment using an EphB4-EphrinB2 inhibitor, or an EphB4-EphrinB2 inhibitor in combination with an immune stimulating drug, for treatment of a number of cancers wherein the subject is currently on a course of treatment with an immune stimulating drug. In some embodiments, the individual suffers from a platinum resistant metastatic cancer. In some embodiments, the individual has not received prior platinum chemotherapy, or is too unhealthy to receive platinum chemotherapy.

In some embodiments, the method of diagnosing and selecting a subject with cancer for treatment using an EphB4-EphrinB2 inhibitor in combination with an immune stimulating drug comprises: i) detecting the level of EphrinB2 expression in a biological sample from a subject who has been diagnosed with a cancer; ii) selecting the subject for treatment using an EphB4-EphrinB2 inhibitor in combination with an immune stimulating drug as front-line therapy when EphrinB2 expression is 1% or greater.

In some embodiments, the method of diagnosing and selecting a subject with cancer for treatment using an EphB4-EphrinB2 inhibitor comprises: i) detecting the level of EphrinB2 expression in a biological sample from a subject who has been diagnosed with a cancer; ii) selecting the subject for treatment using an EphB4-EphrinB2 inhibitor as front-line therapy when EphrinB2 expression is 1% or greater.

In some embodiments, the method of diagnosing a subject with cancer comprises: i) detecting the level of EphrinB2 expression in a biological sample from a subject who has been diagnosed with a cancer and is currently on a course of treatment with an immune stimulating drug; and ii) changing therapy when EphrinB2 expression is 1% or greater.

In some embodiments, EphrinB2 expression is determined by protein expression using a method selected from the group consisting of immunohistochemistry (IHC), immunofluorescence, flow cytometry, and Western blot. In some embodiments, the mRNA expression level is determined using a method selected from the group consisting of quantitative polymerase chain reaction (qPCR), reverse transcription qPCR (RT-qPCR), RNA sequencing, microarray analysis, in situ hybridization, and serial analysis of gene expression (SAGE).

In some embodiments, the biological sample is selected from the group consisting of a tissue sample, a blood sample, a serum sample, a plasma sample, a cerebrospinal fluid (CSF) sample, an ascites fluid sample, and a cell culture sample.

### EphB4-EphrinB2 Inhibitors

Type one receptor tyrosine kinase EphB4 and membrane-localized ligand EphrinB2 induce bidirectional signaling (forward in receptor expressing cells, reverse signaling in ligand expressing cells). EphB4 belongs to the largest family of receptor tyrosine kinases and upon interaction with the EphrinB2 ligand has been reported to regulate neuronal migration, bone remodeling, angiogenesis, cancer progression, and metastasis (Pasquale E B, Cell, 133:38-52, 2008). EphB4 and EphrinB2 expression is downregulated in vast majority of adult normal tissues, even as early as postnatal development but EphB4 is over-expressed in multiple epithelial cancers including lung, bladder, head-neck, and pancreatic cancers (Ferguson B D, et el., Growth Factors, 32:202-6, 2014). Oncogenes including mutant Kras and loss of PTEN induce EphB4 expression. Expression of EphB4 correlates with stage, grade and survival since knock down of EphB4 leads to cell death by apoptosis. The ligand EphrinB2's over-expression and correlation with poor outcome have been reported in several cancer types. ICT increases EphrinB2 in the tumor vessels (and tumor) and high EphrinB2 prevents immune cell recruitment and thus resistance to therapy.

Inhibition of the EphB4-EphrinB2 interaction has a direct inhibitory effect on tumor cell proliferation in vitro and ex-vivo. In various embodiments, the EphB4-Ephrin B2 pathway antagonist or agonist is selected from: (i) a soluble polypeptide comprising the extracellular domain of Ephrin B2; (ii) a soluble polypeptide comprising the extracellular domain of EphB4; (iii) an antibody, or a fragment thereof, that binds to EphB4; (iv) an antibody, or a fragment thereof, that binds to Ephrin B2; (v) a nucleic acid compound that hybridizes to an EphB4 transcript under physiological conditions and decreases the expression of EphB4 in a cell; or (vi) a nucleic acid compound that hybridizes to an EphrinB2 transcript under physiological conditions and decreases the expression of EphrinB2 in a cell.

In various embodiments, the EphB4-Ephrin B2 pathway antagonist inhibits the interaction between Ephrin B2 and EphB4. In various embodiments, the Ephrin B2/EphB4 pathway antagonist inhibits clustering of Ephrin B2 or EphB4. In various embodiments, the EphB4-Ephrin B2 pathway antagonist inhibits phosphorylation of Ephrin B2 or EphB4. In various embodiments, the EphB4-Ephrin B2 pathway agonist stimulates kinase activity of Ephrin B2 or EphB4. In various embodiments, the agent that inhibits EphB4 or EphrinB2 mediated functions is a nucleic acid therapeutic agent. In some embodiments, the nucleic acid therapeutic agent that inhibits EphB4 or EphrinB2 mediated functions is oligonucleotide DNA or siRNA which targets EphrinB2 or EphB4.

Polypeptide agents that inhibit EphB4 or EphrinB2 mediated functions have been previously described by the present inventors (see, e.g., U.S. Pat. Nos. 7,381,410; 7,862,816; 7,977,463; 8,063,183; 8,273,858; 8,975,377; 8,981,062; 9,533,026; each hereby incorporated by reference in their entirety for all purposes). sEphB4-HSA is a fully human fusion protein composed of soluble EphB4 extracellular domain fused at the C-terminus with albumin upon expression as a single seamless protein of 123.3 kDa. sEphB4-HSA specifically binds to EphrinB2. Preliminary studies of sEphB4-HSA in tumor models show increase in T and NK cell migration into tumor. This is accompanied by the induction of ICAM-1 in the tumor vessels. ICAM-1 is an integrin that promotes attachment of T and NK cells to the endothelium followed by transmigration of cells into the tumor. sEphB4-HSA also shows downregulation of PI3K signaling by blocking EphB-EphrinB2 interaction in tumor cell and tumor vessels. sEphB4-HSA blocks the signaling and promote immune cell trafficking into the tumor and inhibit survival signal in tumor cells by downregulating PI3K pathway.

Targeting of EphB4-EphrinB2 represent a therapeutic strategy that has survived the test of clinical trials. It has been shown to be safe in multiple clinical trials with minimal to no toxicity (A. EI-Khoueiry B G, et al., Eur J Cancer, 69, 2016), likely due to low levels of expression in normal tissue. While direct evidence that implicates EphB4-EphrinB2 interaction in the cancer-related immune response is lacking, multiple reports have documented that Eph/ephrin gene family members modulate immune cell processes in inflammatory models, such as arteriosclerosis and wound healing (Braun J, et al., Arterioscler Thromb Vasc Biol, 31:297-305, 2011; Poitz D M, et al., Mol Immunol, 68:648-56, 2015; Yu G, et al., J Immunol, 171:106-14, 2003; Funk S D, et al., Arterioscler Thromb Vasc Biol, 32:686-95, 2012). Eph-ephrin interactions have also been reported to regulate monocyte adhesion to the blood vessel wall trans-endothelial migration, T cell chemotaxis, activation, proliferation and apoptosis, and mobilization of hematopoietic cells from bone marrow sinusoids.

The present inventors have interrogated TCGA database for mRNA expression of EphrinB2 (“EFNB2”). Expression of EFNB2 was significantly higher in tumor tissue compared to normal tissue, based on data analyzed using on comine microarray database.

High-EFNB2 expression correlated significantly with poor survival bladder urothelial cancer (median OS 23.19 vs. 44.28 months). In addition, EFNB2 expression correlated with reduced disease-free survival in the same populations. Verification of the data at protein level remains.

In some embodiments of the present invention, the polypeptide agent that inhibits EphB4 or EphrinB2 mediated functions is a monomeric ligand binding portion of the EphB4 protein or EphrinB2 protein, or an antibody that binds to and affects EphB4 or EphrinB2. In some embodiments, the polypeptide agent is a soluble EphB4 (sEphB4) polypeptide that binds specifically to an EphrinB2 polypeptide and comprises an amino acid sequence of an extracellular domain of an EphB4 protein. In some embodiments, the sEphB4 polypeptide comprises a globular domain of an EphB4 protein. In some embodiments, the agent that inhibits EphB4 or EphrinB2 mediated functions is oligonucleotide DNA or siRNA which targets EphrinB2 or EphB4.

In some embodiments, the sEphB4 polypeptide comprises a sequence selected from the group consisting of a sequence that is at least 90% identical to residues 1-522, at least 90% identical to residues 1-412, and at least 90% identical to residues 1-312 of the amino acid sequence of SEQ ID NO: 1. In some embodiments, the sEphB4 polypeptide may comprise a sequence encompassing the globular (G) domain (amino acids 29-197 of SEQ ID NO; 1), and optionally additional domains, such as the cysteine-rich domain (amino acids 239-321 of SEQ ID NO: 1), the first fibronectin type 3 domain (amino acids 324-429 of SEQ ID NO: 1) and the second fibronectin type 3 domain (amino acids 434-526 of SEQ ID NO: 1). In some embodiments, the sEphB4 polypeptide will comprise amino acids 1-537 of SEQ ID NO: 1. In some embodiments, the sEphB4 polypeptide will comprise amino acids 1-427 of SEQ ID NO: 1. In some embodiments, the sEphB4 polypeptide will comprise amino acids 1-326 of SEQ ID NO: 1. In some embodiments, the sEphB4 polypeptide will comprise amino acids 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537 of SEQ ID NO: 1. In some embodiments, the sEphB4 polypeptide will comprise amino acids 16-197, 16-312, 16-321, 16-326, 16-412, 16-427, 16-429, 16-526 of SEQ ID NO: 1. In some embodiments, a sEphB4 polypeptide may be one that comprises an amino acid sequence at least 90%, and optionally 95% or 99% identical to any of the preceding amino acid sequences while retaining EphrinB2 binding activity. In some embodiments, any variations in the amino acid sequence from the sequence shown in SEQ ID NO: 1 are conservative changes or deletions of no more than 1, 2, 3, 4 or 5 amino acids, particularly in a surface loop region.

In some embodiments, a soluble polypeptide may be prepared in a multimeric form, by, for example, expressing as an Fc fusion protein or fusion with another multimerization domain.

In some embodiments, the sEphB4 polypeptide will further comprise an additional component that confers increased serum half-life while still retaining EphrinB2 binding activity. In some embodiments, the sEphB4 polypeptides are monomeric and are covalently linked to one or more polyoxyaklylene groups (e.g., polyethylene, polypropylene). In some embodiments, the sEphB4 polypeptide is covalently linked to a single polyethylene glycol (PEG) group (hereinafter “sEphB4-PEG”). In some embodiments, the sEphB4 polypeptide is covalently linked to two, three, or more PEG groups.

In some embodiments, the one or more PEG may have a molecular weight ranging from about 1 kDa to about 100 kDa, about 10 to about 60 kDa, and about 10 to about 40 kDa. The PEG group may be a linear PEG or a branched PEG. In some embodiments, the soluble, monomeric sEphB4 conjugate comprises an sEphB4 polypeptide covalently linked to one PEG group of from about 10 to about 40 kDa (monoPEGylated EphB4), or from about 15 to 30 kDa, preferably via an s-amino group of sEphB4 lysine or the N-terminal amino group. In some embodiments, the sEphB4 is randomly PEGylated at one amino group out of the group consisting of the s-amino groups of sEphB4 lysine and the N-terminal amino group.

In some embodiments, the sEphB4 polypeptide is stably associated with a second stabilizing polypeptide that confers improved half-life without substantially diminishing EphrinB2 binding. In some embodiments, the stabilizing polypeptide is immunocompatible with human patients (or animal patients, where veterinary uses are contemplated) and will have little or no significant biological activity. In some embodiments, the sEphB4 polypeptide is associated covalently or non-covalently with an albumin selected from the group consisting of a human serum albumin (HSA) (hereinafter “sEphB4-HSA”) and bovine serum albumin (BSA) (hereinafter “sEphB4-BSA”).

In some embodiments, the covalent attachment may be achieved by expression of the sEphB4 polypeptide as a co-translational fusion with human serum albumin. The albumin sequence may be fused at the N-terminus, the C-terminus or at a non-disruptive internal position in the sEphB4 polypeptide. Exposed loops of the sEphB4 would be appropriate positions for insertion of an albumin sequence. Albumin may also be post-translationally attached to the sEphB4 polypeptide by, for example, chemical cross-linking. In some embodiments, the sEphB4 polypeptide may also be stably associated with more than one albumin polypeptide.

In some embodiments, the sEphB4-HSA fusion inhibits the interaction between EphrinB2 and EphB4, the clustering of EphrinB2 or EphB4, the phosphorylation of EphrinB2 or EphB4, or combinations thereof. In some embodiments, the sEphB4-HSA fusion has enhanced in vivo stability relative to the unmodified wildtype polypeptide.

In some embodiments, the sEphB4-HSA comprises residues 16-197 of SEQ ID NO: 1 directly fused to residues 25-609 of SEQ ID NO: 2. In some embodiments, the sEphB4-HSA comprises residues 16-312 of SEQ ID NO: 1 directly fused to residues 25-609 of SEQ ID NO: 2. In some embodiments, the sEphB4-HSA comprises residues 16-321 of SEQ ID NO: 1 directly fused to residues 25-609 of SEQ ID NO: 2. In some embodiments, the sEphB4-HSA comprises residues 16-326 of SEQ ID NO: 1 directly fused to residues 25-609 of SEQ ID NO: 2. In some embodiments, the sEphB4-HSA comprises residues 16-412 of SEQ ID NO: 1 directly fused to residues 25-609 of SEQ ID NO: 2. In some embodiments, the sEphB4-HSA comprises residues 16-427 of SEQ ID NO: 1 directly fused to residues 25-609 of SEQ ID NO: 2. In some embodiments, the sEphB4-HSA comprises residues 16-429 of SEQ ID NO: 1 directly fused to residues 25-609 of SEQ ID NO: 2. In some embodiments, the sEphB4-HSA comprises residues 16-526 of SEQ ID NO: 1 directly fused to residues 25-609 of SEQ ID NO: 2. In some embodiments, the sEphB4-HSA comprises residues 16-537 of SEQ ID NO: 1 directly fused to residues 25-609 of SEQ ID NO: 2. In some embodiments, the sEphB4-HSA comprises the amino acid sequence set forth in SEQ ID NO: 3. In some embodiments, the sEphB4-HSA comprises the amino acid sequence set forth in SEQ ID NO: 4. In some embodiments, the sEphB4-HSA comprises the amino acid sequence set forth in SEQ ID NO: 5.

In some embodiments, a sEphB4 polypeptide may be prepared in a multimeric form, by, for example, expressing as a fusion protein with a molecule that blocks signaling or blocks EphrinB2 interaction with Eph receptors, including but not limited to antisense oligonucleotides, siRNA, and gene editing like CRISPR/CAS.

In various embodiments, the methods of the present invention may utilize nucleic acid therapeutic agents that inhibit or reduce gene expression of ephrin ligand and/or Eph in a cancer. As used herein, the term “nucleic acid therapeutic agent” or “nucleic acid agent” or “nucleic acid compound” refers to any nucleic acid-based compound that contains nucleotides and has a desired effect on a target gene. Examples of nucleic acid therapeutic agents contemplated for use include, but are not limited to, antisense nucleic acids, dsRNA, siRNA, and enzymatic nucleic acid compounds.

In various embodiments, the disclosure relates to antisense nucleic acids. By “antisense nucleic acid” is meant a non-enzymatic nucleic acid compound that binds to a target nucleic acid by means of RNA-RNA, RNA-DNA or RNA-PNA (protein nucleic acid) interactions and alters the activity of the target nucleic acid (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can form a loop and binds to a substrate nucleic acid which forms a loop. Thus, an antisense molecule can be complementary to two (or more) non-contiguous substrate sequences, or two (or more) non-contiguous sequence portions of an antisense molecule can be complementary to a target sequence, or both. For a review of current antisense strategies, see, e.g., Schmajuk et al., 1999, J. Biol. Chem., 274:21783-21789; Delihas et al., 1997, Nature, 15:751-753; Stein et al., 1997, Antisense N. A. Drug Dev., 7:151; Crooke, 2000, Methods Enzymol., 313:3-45; and Crooke, 1998, Biotech. Genet. Eng. Rev., 15:121-157.

In various embodiments, the antisense nucleic acids of the disclosure can be delivered, for example, as an expression plasmid which, when transcribed in the cell, produces RNA which is complementary to at least a unique portion of the cellular mRNA which encodes an Ephrin B2 or EphB4 polypeptide. Alternatively, the construct is an oligonucleotide which is generated ex vivo and which, when introduced into the cell causes inhibition of expression by hybridizing with the mRNA and/or genomic sequences encoding an Ephrin B2 or EphB4 polypeptide. Such oligonucleotide probes are optionally modified oligonucleotide which are resistant to endogenous nucleases, e.g., exonucleases and/or endonucleases, and are therefore stable in vivo. Exemplary nucleic acid compounds for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

In various embodiments, the disclosure relates to double stranded RNA (dsRNA) and RNAi constructs. The term “dsRNA” as used herein refers to a double stranded RNA molecule capable of RNA interference (RNAi), including siRNA (see, e.g., Bass, 2001, Nature, 411:428-429; Elbashir et al., 2001, Nature, 411:494-498; and Kreutzer et al., PCT Publication No. WO 00/44895; Zernicka-Goetz et al., PCT Publication No. WO 01/36646; and Li et al., PCT Publication No. WO 00/44914). In addition, RNAi is a term initially applied to a phenomenon observed in plants and worms where double-stranded RNA (dsRNA) blocks gene expression in a specific and post-transcriptional manner. RNAi provides a useful method of inhibiting gene expression in vitro or in vivo.

The term “short interfering RNA,” “siRNA,” or “short interfering nucleic acid,” as used herein, refers to any nucleic acid compound capable of mediating RNAi or gene silencing when processed appropriately be a cell. For example, the siRNA can be a double-stranded polynucleotide molecule comprising self-complementary sense and antisense regions, wherein the antisense region comprises complementarity to a target nucleic acid compound (e.g., Ephrin B2 or EphB4). The siRNA can be a single-stranded hairpin polynucleotide having self-complementary sense and antisense regions, wherein the antisense region comprises complementarity to a target nucleic acid compound. The siRNA can be a circular single-stranded polynucleotide having two or more loop structures and a stem comprising self-complementary sense and antisense regions, wherein the antisense region comprises complementarity to a target nucleic acid compound, and wherein the circular polynucleotide can be processed either in vivo or in vitro to generate an active siRNA capable of mediating RNAi. The siRNA can also comprise a single stranded polynucleotide having complementarity to a target nucleic acid compound, wherein the single stranded polynucleotide can further comprise a terminal phosphate group, such as a 5′-phosphate (see for example Martinez et al., 2002, Cell., 110, 563-574), or 5′,3′-diphosphate.

Applicants have generated a number of monoclonal antibodies against EphB4 as well as hybridoma cell lines producing EphB4 monoclonal antibodies. These antibodies were further characterized in many ways, such as, their ability to inhibit interaction between EphB4 and its ligand (e.g., Ephrin B2), their ability to inhibit dimerization or multimerization of EphB4 receptor, their ability to induce tyrosine phosphorylation of EphB4, their cross-reactivity with other Eph family members, their ability to inhibit angiogenesis, and their ability to inhibit tumor growth. Further, epitope mapping studies reveals that these EphB4 antibodies may specifically bind to one or more regions of EphB4 (e.g., a globular domain, a cystein-rich domain, or a fibronectin type Ill domain). For example, an EphB4 antibody may bind to both fibronectin type 3 domains. Such antibodies have been described in, e.g., US 20050249736, US 20090196880, U.S. Pat. Nos. 8,273,858, 8,981,062 and 8,975,377.

In various embodiments, methods of the present invention include administering to a patient in need of treatment a therapeutically effective amount or an effective dose of sEphB4-HSA polypeptide of the present invention. In various embodiments, effective doses of the polypeptides of the present invention, e.g., for the treatment of primary or metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In various embodiments, the dosage may range from about 0.0001 to 100 mg/kg, and more usually 0.01 to 5.0 mg/kg, of the host body weight. For example, dosages can be 1 mg/kg body weight or 10 mg/kg body weight or within the range of 1-10 mg/kg. In various embodiments, the dosage of the polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 6.0, of about 7.0, of about 8.0, of about 9.0, and of about 10.0 mg/kg. In various embodiments, the treatment regime entails administration once per every two weeks or once a month or once every 3 to 6 months. Therapeutic entities of the present invention are usually administered on multiple occasions. Intervals between single dosages can be weekly, bi-weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

### Immune Stimulating Drug

A number of immune-checkpoint protein antigens have been reported to be expressed on various immune cells, including, e.g., SIRP (expressed on macrophage, monocytes, dendritic cells), CD47 (highly expressed on tumor cells and other cell types), VISTA (expressed on monocytes, dendritic cells, B cells, T cells), TIGIT (immune receptor present on some T cells and natural killer cells), CD152 (expressed by activated CD8+ T cells, CD4+ T cells and regulatory T cells), CD279 (expressed on tumor infiltrating lymphocytes, expressed by activated T cells (both CD4 and CD8), regulatory T cells, activated B cells, activated NK cells, anergic T cells, monocytes, dendritic cells), CD274 (expressed on T cells, B cells, dendritic cells, macrophages, vascular endothelial cells, pancreatic islet cells), and CD223 (expressed by activated T cells, regulatory T cells, anergic T cells, NK cells, NKT cells, and plasmacytoid dendritic cells)(see, e.g., Pardoll, D., Nature Reviews Cancer, 12:252-264, 2012). LAG3, TIM3, TIGIT, OX40 ligand, interferons, and IL-2.

Antibodies that bind to an antigen which is determined to be an immune-checkpoint protein are known to those skilled in the art. For example, various anti-CD276 antibodies have been described in the art (see, e.g., U.S. Pat. Public. No. 20120294796 (Johnson et al) and references cited therein); various anti-CD272 antibodies have been described in the art (see, e.g., U.S. Pat. Public. No. 20140017255 (Mataraza et al) and references cited therein); various anti-CD152/CTLA-4 antibodies have been described in the art (see, e.g., U.S. Pat. Public. No. 20130136749 (Korman et al) and references cited therein); various anti-LAG-3/CD223 antibodies have been described in the art (see, e.g., U.S. Pat. Public. No. 20110150892 (Thudium et al) and references cited therein); various anti-CD279 (PD-1) antibodies have been described in the art (see, e.g., U.S. Pat. No. 7,488,802 (Collins et al) and references cited therein); various anti-PD-L1 antibodies have been described in the art (see, e.g., U.S. Pat. Public. No. 20130122014 (Korman et al) and references cited therein); various anti-TIM-3 antibodies have been described in the art (see, e.g., U.S. Pat. Public. No. 20140044728 (Takayanagi et al) and references cited therein); and various anti-B7-H4 antibodies have been described in the art (see, e.g., U.S. Pat. Public. No. 20110085970 (Terrett et al) and references cited therein). Each of these references is hereby incorporated by reference in its entirety for the specific antibodies and sequences taught therein.

Immune checkpoint inhibitor targeting agents are effective in patients with tumors that express inflammatory signatures and contain resident immune cells commonly referred to as “hot tumors.” Tumors with a few or no immune cells (cold tumors) are less likely or unlikely to respond. Immune checkpoint PD-1 and CTLA inhibitors are efficacious in several cancers which express interferon gamma signature, are rich in tumor infiltrating immune cells and express PD-L1. Tumor vessels regulate immune cell exit into the tumors, thus tumor vessel modulation may offer avenue to change tumor environment.

The PD-1 receptor-ligand interaction is a major pathway hijacked by tumors to suppress immune control. The normal function of PD-1, expressed on the cell surface of activated T-cells under healthy conditions, is to down-modulate unwanted or excessive immune responses, including autoimmune reactions. The ligands for PD-1 (PD-L1 and PD-L2) are constitutively expressed or can be induced in a variety of cell types, including non-hematopoietic tissues as well as in various tumors. Binding of either PD-1 ligand to PD-1 inhibits T-cell activation triggered through the T-cell receptor. PD-1 has been suggested to regulate tumor-specific T-cell expansion in subjects with melanoma (MEL). This suggests that the PD-1/PD-L1 pathway plays a critical role in tumor immune evasion and should be considered as an attractive target for therapeutic intervention.

Pembrolizumab (KEYTRUDA®) is a potent and highly selective humanized monoclonal antibody (mAb) of the IgG4/kappa isotype designed to directly block the interaction between PD-1 and its ligands, PD-L1 and PD-L2. The Food and Drug Administration (FDA) approved KEYTRUDA® on Aug. 5, 2016, for the treatment of some patients with an advanced form of head and neck cancer. The approval is for patients with recurrent or metastatic head and neck squamous cell carcinoma (HNSCC) that has continued to progress despite standard-of-care treatment with chemotherapy. KEYTRUDA® has recently been approved in the United Stated for the treatment of patients with unresectable or metastatic melanoma and disease progression following ipilumumab and, if BRAF V600 mutation positive, a BRAF inhibitor.

Nivolumab (OPDIVO®) is a human IgG4 anti-PD-1 monoclonal antibody that works as a checkpoint inhibitor, blocking a signal that would have prevented activated T cells from attacking the cancer, thus allowing the immune system to clear the cancer. OPDIVO® is used as a first line treatment for inoperable or metastatic melanoma in combination with ipilimumab if the cancer does not have a mutation in BRAF as a second-line treatment following treatment with ipilimumab and if the cancer has a mutation in BRAF, with a BRAF inhibitor as a second-line treatment for squamous non-small cell lung cancer and as a second-line treatment for renal cell carcinoma.

In various embodiments, the PD-1 inhibitor used in the combination therapy methods is selected from the group consisting of, but not limited to, nivolumab (Bristol-Myers Squibb)(Drugbank 09035; Drugbank 06132), pembrolizumab (Merck)(Drugbank 09037) and pidilizumab (Medivation)(Drugbank 15383).

In various embodiments, the CTLA-4 inhibitor is selected from the group consisting of, but not limited to, ipilimumab (Bristol-Myers Squibb)(Drugbank 06186) and tremelimumab (Medlmmune)(Drugbank 11771).

### Cancers

Urothelial carcinoma with an incidence of 80,470 cases per year causes 17,670 deaths per year and remains a significant health challenge in the United States (Siegel R L, et al., Cancer J Clin. 2019; 69(1):7-34, 2019). If untreated, the patients have a median survival of ˜4.5 months. If treated with cytotoxic chemotherapy, the survival increased to ˜7.5 months with ORR of ˜15% and PFS of 3-3.5 months. Cytotoxic chemotherapy however results in substantial toxicity. Combination cytotoxic chemotherapy results in modestly improved response rates without improvement in survival, but worsened toxicity. Consequently, before the advent of immunotherapy, for previously treated metastatic urothelial patients, monotherapy has been favored over combination therapy. Most commonly used single agents in the US included gemcitabine, paclitaxel, and docetaxel.

In the metastatic setting the standard of care in the frontline setting has not changed since 2000 and remains cisplatin-based chemotherapy. A number of single agents such as vinflunine (ORR 18%, OS 6.6 months), gemcitabine (ORR 11%, OS 8.7 months), pemetrexed (ORR 28%, OS 9.6 months), paclitaxel (ORR 10%, OS 7.2 months) and combination regimens such as paclitaxel with methotrexate (ORR 32%, OS 5 months) or gemcitabine (ORR 47%, OS 7.5 months) or docetaxel with ifosfamide (ORR 25%, OS 4 months) have been studied after failure of first line therapy. Based on the safety and efficacy, most commonly used agents are paclitaxel, docetaxel, and carboplatin. Response rates are ˜10-15% and overall survival of 6-9 months. Combination chemotherapy resulted in higher response rate, greater toxicity, but without improvement in survival (Raggi D, et al., Ann Oncol. 27(1):49-61, 2016). It was not until the approval of anti-PD1/PDL1 antibodies that a more durable second line option with survival benefit became available for previously treated patients with metastatic urothelial carcinoma. With an expected median survival of up to 10.3 months and response rate of 21.1%, 5 different drugs are in clinical practice including pembrolizumab, nivolumab, atezolizumab, avelumab, and durvalumab. Pembrolizumab is approved for this patient population, it is effective in only a minority of the patients having a median overall survival (OS) of 10.3 months (95% Cl, 8-11.8), median overall progression free survival (PFS) of 2.1 months (95% Cl, 2.0-2.2), with overall response rate (ORR) of 21.1% (95% Cl, 16.4 to 26.5), and complete response rate of 7% in this patient population.

Hepatocellular carcinoma (HCC) is the most frequent cancer in certain parts of the world, and the fifth most cancer common worldwide. Globally, it is the second leading cause of cancer death in men and the sixth leading cause of cancer death among women (see, e.g., Parkin D. M., Lancet Oncology, 2:533-43, 2001). Because HCC is often diagnosed late in the course of clinical manifestation, only 10-15% of patients are candidates for curative surgery. For the majority of HCC patients, systemic chemotherapies or supportive therapies are the mainstay treatment options. HCC in general is highly refractory to therapy and most chemotherapeutic agents show limited effectiveness and have not been able to improve patient survival (see, e.g., Gish R. G. et al., J. of Clinical Oncology 25:3069-75, 2007; Ramanathan R. K. et al., J. of Clinical Oncology 24:4010, 2006). Recent studies evaluating the Programmed Death 1 (PD-1) antibody nivolumab (OPDIVO®) showed response rates of around 10-20%. Response duration was 14-17+ months for CR, <1-8+ months for PR, and 1.5-17+ months for stable disease (SD). Overall survival (OS) rate at 6 months is 72%. Nivolumab demonstrated a manageable AE profile and produced durable responses across all dose levels and HCC cohorts, with a favorable 6-month OS rate.

Head and neck squamous cell carcinoma (HNSCC) accounts for almost 90% of cancers involving the upper aerodigestive tract (UADT). In the United States in 2005, cancers of the oral cavity, pharynx and larynx are expected to account for nearly 3% of incident cancers and 2% of cancer deaths. There are approximately 500,000 new cases diagnosed world-wide each year. Men are affected over two times more than women. Over half of these cancers involve the oral cavity. The rest are divided equally between larynx and pharynx. Numerous clinical trials are testing the benefits of immunotherapy in human cancer, including head and neck squamous cell carcinoma (HNSCC). The objective response rate is 6-20% (Szturz P, et al., BMC Med, 15:110, 2017; Ferris R L, et al., Oral Oncol, 81:45-51, 2018; Postow M A, et al., J Clin Oncol, 33:1974-82, 2015; Chow L Q M, et al., J Clin Oncol, 34:3838-45, 2016; Siu L L, et al., JAMA Oncol 2018) and the vast majority of patients demonstrate either innate or adaptive resistance to immunotherapy. Attempts at simply combining more immune checkpoint inhibitors have also proven disappointing due to increased toxicity to patients and lack of additional benefit (https://clinicaltrials.gov/ct2/show/NCT02205333). In orthotopic mouse models of HNSCC, we have recently demonstrated that tumor regrowth occurs even after combination treatment with anti-PDL1 antibody and radiation therapy (RT) (7,8). Oweida A, et al., Clin Cancer Res, 2018; Messenheimer D J, et al., Clin Cancer Res, 23:6165-77, 2017).

Radiation therapy remains the standard of care treatment in the definitive management of patients with locally advanced HNSCCs and can act as an adjuvant for immunotherapy but there are some undesirable effects mounted in response to RT that in turn compromises the efficacy of immunotherapeutic agents. RT is unable to overcome the accumulation of immunosuppressive populations such as Tregs in the later (repair) phase (7). Therefore, finding other treatments that synergize with RT and counteract its negative effects is critical to overcome adverse side-effects, treatment resistance, and tumor regrowth.

Five-year survival rates for HNSCC are low and have not improved in several decades. Moreover, patients with this disease experience severe morbidity including disfigurement, speech, swallowing and breathing problems. Late stage of diagnosis and propensity to recur are challenges that thwart efforts to improve outcomes in these patients. Pembrolizumab is a potent and highly selective humanized monoclonal antibody (mAb) of the IgG4/kappa isotype designed to directly block the interaction between PD-1 and its ligands, PD-L1 and PD-L2. The Food and Drug Administration (FDA) approved pembrolizumab (KEYTRUDA®) on Aug. 5, 2016, for the treatment of some patients with an advanced form of head and neck cancer. The approval is for patients with recurrent or metastatic head and neck squamous cell carcinoma (HNSCC) that has continued to progress despite standard-of-care treatment with chemotherapy. According to the FDA approval summary, 28 patients (16%) experienced a tumor response following treatment with pembrolizumab. In 23 (82%) of those patients, the tumor response lasted for 6 months or longer, and several have lasted for more than 2 years. Patients with HNSCC whose tumors are positive for the human papillomavirus (HPV) typically have better outcomes after treatment with chemotherapy than patients whose tumors are HPV negative. According to the FDA approval summary, responses were seen in patients with HPV-positive tumors as well as in patients with HPV-negative tumors (24% and 16%, respectively).

Non-small cell lung cancer (NSCLC) is the most common type of lung cancer. Squamous cell carcinoma, adenocarcinoma, and large cell carcinoma are all subtypes of NSCLC. NSCLC accounts for about 85% of all lung cancers. As a class, NSCLCs are relatively insensitive to chemotherapy, compared to small cell carcinoma. When possible, they are primarily treated by surgical resection with curative intent, although chemotherapy is increasingly being used both pre-operatively (neoadjuvant chemotherapy) and post-operatively (adjuvant chemotherapy). On Oct. 2, 2015, the FDA approved pembrolizumab for the treatment of metastatic non-small cell lung cancer (NSCLC) in patients whose tumors express PD-L1 and who have failed treatment with other chemotherapeutic agents. In October 2016, pembrolizumab became the first immunotherapy to be used first line in the treatment of NSCLC if the cancer overexpresses PDL1 and the cancer has no mutations in EGFR or in ALK; if chemotherapy has already been administered, then pembrolizumab can be used as a second line treatment but if the cancer has EGFR or ALK mutations, agents targeting those mutations should be used first. Assessment of PDL1 must be conducted with a validated and approved companion diagnostic. In the Keynote-001 trial (NTC01295827), the efficacy and safety of programmed cell death 1 (PD-1) inhibition with pembrolizumab was assessed in patients with advanced non-small-cell lung cancer. Among all the patients, the objective response rate was 19.4%, and the median duration of response was 12.5 months. The median duration of progression-free survival was 3.7 months, and the median duration of overall survival was 12.0 months. PD-L1 expression in at least 50% of tumor cells was selected as the cutoff from the training group. Among patients with a proportion score of at least 50% in the validation group, the response rate was 45.2%. Among all the patients with a proportion score of at least 50%, median progression-free survival was 6.3 months; median overall survival was not reached. PD-L1 expression in at least 50% of tumor cells correlated with improved efficacy of pembrolizumab (Garon et al., N Engl J Med, 372:2018-2028, 2015).

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens, such as testosterone, promote prostate cancer tumor growth. At its early stages, localized prostate cancer is often treated with local therapy including, for example, surgical removal of the prostate gland and radiotherapy. However, when local therapy fails to cure prostate cancer, as it does in up to a third of men, the disease progresses into incurable metastatic disease (i.e., disease in which the cancer has spread from one part of the body to other parts). As used herein, the term “prostate cancer” is used in the broadest sense and refers to all stages and all forms of cancer arising from the tissue of the prostate gland. The term “prostate cancer” encompasses any type of malignant (i.e. non-benign) tumor located in prostatic tissues, such as e.g. prostatic adenocarcinoma, prostatic sarcoma, undifferentiated prostate cancer, prostatic squamous cell carcinoma, prostatic ductal transitional carcinoma and prostatic intraepithelial neoplasia.

Kaposi sarcoma (KS) is a multifocal angioproliferative disorder of vascular endothelium, most associated with infection with the Kaposi-sarcoma associated herpes virus (KSHV), also known as human herpes virus-8 (HHV-8). KS is associated with a number of epidemiologic and pathophysiologic factors. KS is classified into four distinct clinical types: classic Mediterranean KS, African-endemic KS, immunosuppressive drug-related KS, and HIV-related KS. A rare disease before the era of HIV and AIDS, HIV-related KS is the most frequent malignancy in HIV infected patients. KS can affect many organs. KS manifests most frequently as a disease of the skin. In many advanced cases, KS involves organs such as the lungs, liver, or gastrointestinal tract. At this time, KS is incurable. Available therapies are for palliation. Systemic chemotherapy is generally used for patients with more advanced disease or evidence of rapid progression of disease. The major goals of treatment are symptom palliation, prevention of disease progression, and reduction of tumor burden to alleviate lymphedema, organ compromise, and psychological stress. The standard therapies for visceral or advanced cutaneous KS include cytotoxic chemotherapy such as liposomal anthracycline and paclitaxel. Liposomal doxorubicin has superior efficacy and favorable tolerability and toxicity compared to the combination of non-liposomal doxorubicin, vincristine, and bleomycin with overall response rates of 59% in HIV patients. In classical KS, response rates to liposomal doxorubicin can be higher. However, complete response rates are uncommon and there is no cure. At this point in time, no targeted therapy has been fully developed for KS.

In 2014, it is projected that 46,420 new cases of pancreatic cancer will be diagnosed in the United States, with an estimated 39,590 deaths from the disease. Although surgical resection is the only potentially curative treatment modality, only 15-20% of patients have respectable disease at diagnosis, and the treatment for unresectable, locally advanced, and metastatic pancreatic cancer remains largely palliative. Gemcitabine monotherapy has been used as the reference regimen for treatment of advanced pancreatic cancer after a randomized trial showed a clinical benefit as well as a survival benefit of about one month when compared to single-agent fluorouracil. Combination therapy with gemcitabine-based regimens for locally advanced and metastatic pancreatic cancer was shown in a meta-analysis to provide a slight benefit in overall survival (OS), albeit with more frequent toxicities, and there is also evidence to suggest an improved benefit with combination regimens in patients with good performance status. One such combination regimen is gemcitabine plus albumin-bound paclitaxel (nab-paclitaxel). In the phase 3 open-label MPACT trial, 861 patients were randomized in a 1:1 ratio to receive either intravenous infusions of gemcitabine (1000 mg per square meter body surface area or mg/m²) alone or gemcitabine (1000 mg/m²) plus nab-paclitaxel (125 mg/m²). The combination group had an increased median overall survival of 8.5 months as compared to 6.7 months in the single agent group, but more high-grade neutropenia, fatigue, and neuropathy were seen in the former. In the combination group, 41% of patients had dose-reductions of nab-paclitaxel, and 47% had dose reductions of gemcitabine. Because of the 1.8 month increase in OS, this study led to the 2013 Food and Drug Administration (FDA) approval of nab-paclitaxel for the treatment of late-stage pancreatic cancer. An updated OS analysis of the MPACT study published in 2015 confirmed a longer median OS of 8.7 months in the nab-paclitaxel and gemcitabine combination group, compared to 6.6 months in the gemcitabine monotherapy group.

In various embodiments, the cancer is selected from the group consisting of, but not limited to, non-small cell lung carcinoma (NSCLC), colon carcinoma, metastatic urothelial cancer, breast cancer, hepatocellular carcinoma (HCC), mesothelioma, pancreatic cancer, prostate cancer, bladder cancer, squamous cell carcinoma of the head and neck (HNSCC), Kaposi sarcoma, and leukemia.

In various embodiments, the patient previously responded to treatment with an anti-cancer therapy, but, upon cessation of therapy, suffered relapse (hereinafter “a recurrent proliferative disease”).

In various embodiments, the patient has resistant or refractory cancer. In various embodiments, the cancer is refractory to immunotherapy treatment. In various embodiments, the cancer is refractory to treatment with a chemotherapeutic agent. In various embodiments, the cancer is refractory to treatment using depleting antibodies to specific tumor antigens. In various embodiments, the cancer is refractory to treatment using agonistic, antagonistic, or blocking antibodies to co-stimulatory or co-inhibitory molecules (immune checkpoints). In various embodiments, the cancer is refractory to targeted treatment with an immunoconjugate, antibody-drug conjugate (ADC), or fusion molecule comprising a depleting antibody to a specific tumor antigen and a cytotoxic agent. In various embodiments, the cancer is refractory to targeted treatment with a small molecule kinase inhibitor. In various embodiments, the cancer is refractory to combination therapy involving, for example, two or more of: immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), treatment with a immunoconjugate, ADC, or fusion molecule comprising a depleting antibody to a specific tumor antigen and a cytotoxic agent, targeted treatment with a small molecule kinase inhibitor, treatment using surgery, treatment using a therapeutic vaccine, treatment using stem cell transplantation, and treatment using radiation.

### Combination Therapy

In some embodiments, the method for treating or delaying progression of a cancer in a subject further comprises a second therapy selected from the group consisting of: small molecule kinase inhibitor targeted therapy, surgery, cytoreductive therapy, cytotoxic chemotherapy, and immunotherapy. In some embodiments, the combination therapy will be synergistic. In some embodiments, the second therapy is cytoreductive therapy and the combination may increase the therapeutic index of the cytoreductive therapy. In some embodiments, the cytoreductive therapy may act in a DNA repair pathway. In some embodiments, the cytoreductive therapy is radiation therapy. In some embodiments, the combination may be synergistic.

In some embodiments, the combination therapy comprises anti-proliferative, or cytoreductive therapy. Anti-proliferative, or cytoreductive therapy is used therapeutically to eliminate tumor cells and other undesirable cells in a host and includes the use of therapies such as delivery of ionizing radiation, and administration of chemotherapeutic agents. For example, ionizing radiation (IR) is used to treat about 60% of cancer patients, by depositing energy that injures or destroys cells in the area being treated, and for the purposes of the present invention may be delivered at conventional doses and regimens, or at reduced doses. Radiation injury to cells is nonspecific, with complex effects on DNA. The efficacy of therapy depends on cellular injury to cancer cells being greater than to normal cells.

Radiotherapy may be used to treat every type of cancer. Some types of radiation therapy involve photons, such as X-rays or gamma rays. Another technique for delivering radiation to cancer cells is internal radiotherapy, which places radioactive implants directly in a tumor or body cavity so that the radiation dose is concentrated in a small area. A suitable dose of ionizing radiation may range from at least about 2 Gy to not more than about 10 Gy, usually about 5 Gy. A suitable dose of ultraviolet radiation may range from at least about 5 J/m² to not more than about 50 J/m², usually about 10 J/m². The sample may be collected from at least about 4 and not more than about 72 hours following ultraviolet radiation, usually around about 4 hours.

Chemotherapeutic agents are well-known in the art and are used at conventional doses and regimens, or at reduced dosages or regimens, including for example, topoisomerase inhibitors such as anthracyclines, including the compounds daunorubicin, adriamycin (doxorubicin), epirubicin, idarubicin, anamycin, MEN 10755, and the like. Other topoisomerase inhibitors include the podophyllotoxin analogues etoposide and teniposide, and the anthracenediones, mitoxantrone and amsacrine. Other anti-proliferative agent interferes with microtubule assembly, e.g., the family of vinca alkaloids. Examples of vinca alkaloids include vinblastine, vincristine; vinorelbine (NAVELBINE); vindesine; vindoline; vincamine; etc. DNA-damaging agent include nucleotide analogs, alkylating agents, etc. Alkylating agents include nitrogen mustards, e.g., mechlorethamine, cyclophosphamide, melphalan (L-sarcolysin), etc.; and nitrosoureas, e.g., carmustine (BCNU), lomustine (CCNU), semustine (methyl-CCNU), streptozocin, chlorozotocin, etc. Nucleotide analogs include pyrimidines, e.g., cytarabine (CYTOSAR-U), cytosine arabinoside, fluorouracil (5-FU), floxuridine (FUdR), etc.; purines, e.g., thioguanine (6-thioguanine), mercaptopurine (6-MP), pentostatin, fluorouracil (5-FU) etc.; and folic acid analogs, e.g., methotrexate, 10-propargyl-5,8-dideazafolate (PDDF, CB3717), 5,8-dideazatetrahydrofolic acid (DDATHF), leucovorin, etc. Other chemotherapeutic agents of interest include metal complexes, e.g., cisplatin (cis-DDP), carboplatin, oxaliplatin, etc.; ureas, e.g., hydroxyurea; gemcitabine, and hydrazines, e.g., N-methylhydrazine. In some embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

In some embodiments, the combination therapy will comprise immunotherapy. As used herein, the term “immunotherapy” refers to cancer treatments which include, but are not limited to treatment using depleting antibodies to specific tumor antigens (see, e.g., reviews by Blattman and Greenberg, Science, 305:200, 2004; Adams and Weiner, Nat Biotech, 23:1147, 2005; Vogal et al. J Clin Oncology, 20:719, 2002; Colombat et al., Blood, 97:101, 2001); treatment using antibody-drug conjugates (see, e.g., Ducry, Laurent (Ed.) Antibody Drug Conjugates. In: Methods in Molecular Biology. Book 1045. New York (NY), Humana Press, 2013; Nature Reviews Drug Discovery 12, 259-260, April 2013); treatment using agonistic, antagonistic, or blocking antibodies to co-stimulatory or co-inhibitory molecules (immune checkpoints) such as CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), OX-40, CD137, GITR, LAG3, TIM-3, and VISTA (see, e.g., Sharon et al., Chin J Cancer., 33(9): 434-444, September 2014; Hodi et al., N Engl J Med, 2010; Topalian et al., N Engl J Med, 366:2443-54, 2012); treatment using bispecific T cell engaging antibodies (BiTE®) such as blinatumomab (see, e.g., U.S. Pat. No. 9,260,522; US Patent Application No. 20140302037); treatment involving administration of biological response modifiers such as IL-2, IL-12, IL-15, IL-21, GM-CSF, IFN-α, IFN-β, and IFN-γ (see, e.g., Sutlu T et al., Journ of Internal Medicine, 266(2):154-181, 2009; Joshi S PNAS USA, 106(29):12097-12102, 2009; Li Y et al., Journal of Translational Medicine, 7:11, 2009); treatment using therapeutic vaccines such as sipuleucel-T (see, e.g., Kantoff P W New England Journal of Medicine, 363(5):411-422, 2010; Schlom J., Journal of the National Cancer Institutes, 104(8):599-613, 2012); treatment using dendritic cell vaccines, treatment using oncolytic virus therapy (e.g., T-VEC); treatment using tumor antigen peptide vaccines; treatment using chimeric antigen receptor (CAR)-T cells (see, e.g., Rosenberg S A Nature Reviews Cancer, 8(4):299-308, 2008; Porter D L et al, New England Journal of Medicine, 365(8):725-733, 2011; Grupp S A et al., New England Journal of Medicine, 368(16):1509-151, 2013; U.S. Pat. Nos. 9,102,761; 9,101,584); treatment using CAR-NK cells (see, e.g., Glienke et al., Front Pharmacol, 6(21):1-7, February 2015); treatment using tumor infiltrating lymphocytes (TILs)(see e.g., Wu et al, Cancer J., 18(2): 160-175, 2012); treatment using adoptively transferred anti-tumor T cells (ex vivo expanded and/or TCR transgenic)(see e.g., Wrzesinski et al., J Immunother, 33(1): 1-7, 2010); treatment using TALL-104 cells; and treatment using immunostimulatory agents such as Toll-like receptor (TLR) agonists CpG and imiquimod (see, e.g., Krieg, Oncogene, 27:161-167, 2008; Lu, Front Immunol, 5(83):1-4, March 2014).

Immunotherapy focused on utilization of depleting antibodies to specific tumor antigens have been explored with much success (see, e.g., reviews by Blattman and Greenberg, Science, 305:200, 2004; Adams and Weiner, Nat Biotech, 23:1147, 2005). A few examples of such tumor antigen-specific, depleting antibodies are HERCEPTIN® (anti-Her2/neu mAb)(Baselga et al., J Clin Oncology, Vol 14:737, 1996; Baselga et al., Cancer Research, 58:2825, 1998; Shak, Semin. Oncology, 26 (Suppl12):71, 1999; Vogal et al. J Clin Oncology, 20:719, 2002); and RITUXAN® (anti-CD20 mAb)(Colombat et al., Blood, 97:101, 2001). Unfortunately, while clearly having made a mark in oncology treatment, as monotherapy they generally work in only about 30% of the individuals and with a partial response. Moreover, many individuals eventually become refractory or relapse after treatment with these antibody-containing regimens.

Treatment using agonistic, antagonistic, or blocking antibodies to co-stimulatory or co-inhibitory molecules (immune checkpoints) has been an area of extensive research and clinical evaluation. Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll D M., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), and OX-40, CD137, GITR, LAG3, TIM-3, and VISTA (see, e.g., Sharon et al., Chin J Cancer., 33(9): 434-444, September 2014; Hodi et al., N Engl J Med, 2010; Topalian et al., N Engl J Med, 366:2443-54) are being evaluated as new, alternative immunotherapies to treat patients with proliferative diseases such as cancer, and in particular, patients with refractory and/or recurrent cancers.

In various embodiments, between about 0.1 mg/kg to about 10 mg/kg of PD-1 inhibitor is administered. In various embodiments, between about 1 mg/kg to about 15 mg/kg of PD-1 inhibitor is administered. In various embodiments, between about 3 mg/kg to about 12 mg/kg of PD-1 inhibitor is administered. In various embodiments, between about 1 mg/kg to about 10 mg/kg of PD-1 inhibitor is administered. In various embodiments, between about 3 mg/kg to about 10 mg/kg of PD-1 inhibitor is administered. In various embodiments, at least about 1 mg/kg of PD-1 inhibitor is administered. In various embodiments, at least about 2 mg/kg of PD-1 inhibitor is administered. In various embodiments, at least about 3 mg/kg of PD-1 inhibitor is administered. In various embodiments, at least about 5 mg/kg of PD-1 inhibitor is administered. In various embodiments, at least about 10 mg/kg of PD-1 inhibitor is administered. In various embodiments, between about 10 mg to about 400 mg of PD-1 inhibitor is administered. In various embodiments, between about 50 mg to about 400 mg of PD-1 inhibitor is administered. In various embodiments, between about 10 mg to about 300 mg of PD-1 inhibitor is administered. In various embodiments, between about 50 mg to about 300 mg of PD-1 inhibitor is administered. In various embodiments, between about 10 mg to about 250 mg of PD-1 inhibitor is administered. In various embodiments, between about 50 mg to about 250 mg of PD-1 inhibitor is administered. In various embodiments, at least about 50 mg of PD-1 inhibitor is administered. In various embodiments, at least about 100 mg of PD-1 inhibitor is administered. In various embodiments, at least about 150 mg of PD-1 inhibitor is administered. In various embodiments, at least about 200 mg of PD-1 inhibitor is administered. In various embodiments, at least about 250 mg of PD-1 inhibitor is administered. In various embodiments, at least about 300 mg of PD-1 inhibitor is administered. In various embodiments, the PD-1 inhibitor is administered at least once during a cycle. In various embodiments, the PD-1 inhibitor is administered at least twice during a cycle. In various embodiments, a cycle is 21 days. In various embodiments, a cycle is 28 days. In various embodiments, the PD-1 inhibitor is administered at least once a week. In various embodiments, the PD-1 inhibitor is administered at least once every two weeks. In various embodiments, the PD-1 inhibitor is administered at least once every three weeks. In various embodiments, the PD-1 inhibitor is administered at least once every four weeks.

Treatment using chimeric antigen receptor (CAR) T cell therapy is an immunotherapy in which the patient's own T cells are isolated in the laboratory, redirected with a synthetic receptor to recognize a particular antigen or protein, and reinfused into the patient. CARs are synthetic molecules that minimally contain: (1) an antigen-binding region, typically derived from an antibody, (2) a transmembrane domain to anchor the CAR into the T cells, and (3) 1 or more intracellular T cell signaling domains. A CAR redirects T cell specificity to an antigen in a human leukocyte antigen (HLA)-independent fashion, and overcomes issues related to T cell tolerance (Kalos M and June C H, Immunity, 39(1):49-60, 2013). Over the last 5 years, at least 15 clinical trials of CAR-T cell therapy have been published. A new wave of excitement surrounding CAR-T cell therapy began in August 2011, when investigators from the University of Pennsylvania (Penn) published a report on 3 patients with refractory chronic lymphocytic leukemia (CLL) who had long-lasting remissions after a single dose of CAR T cells directed to CD 19 (Porter D L, et al., N Engl J Med., 365(8):725-733, 2011).

In contrast to donor T cells, natural killer (NK) cells are known to mediate anti-cancer effects without the risk of inducing graft-versus-host disease (GvHD). Accordingly, alloreactive NK cells are now also the focus of considerable interest as suitable and powerful effector cells for cellular therapy of cancer. Several human NK cell lines have been established, e.g., NK-92, HANK-1, KHYG-1, NK-YS, NKG, YT, YTS, NKL and NK3.3 (Kornbluth, J., et al., J. Immunol. 134, 728-735, 1985; Cheng, M. et al., Front. Med. 6:56, 2012) and various CAR expressing NK cells (CAR-NK) have been generated. Immunotherapy using CAR expressing NK cells (CAR-NK) is an active area of research and clinical evaluation (see, e.g., Glienke et al., Front Pharmacol, 6(21):1-7, February 2015).

Bispecific T-cell engager molecules (BiTE®s) constitute a class of bispecific single-chain antibodies for the polyclonal activation and redirection of cytotoxic T cells against pathogenic target cells. BiTE®s are bispecific for a surface target antigen on cancer cells, and for CD3 on T cells. BiTE®s are capable of connecting any kind of cytotoxic T cell to a cancer cell, independently of T-cell receptor specificity, costimulation, or peptide antigen presentation. a unique set of properties that have not yet been reported for any other kind of bispecific antibody construct, namely extraordinary potency and efficacy against target cells at low T-cell numbers without the need for T-cell co-stimulation (Baeuerle et al., Cancer Res, 69(12):4941-4, 2009). BiTE antibodies have so far been constructed to more than 10 different target antigens, including CD19, EpCAM, Her2/neu, EGFR, CD66e (or CEA, CEACAM5), CD33, EphA2, and MCSP (or HMW-MAA)(Id.) Treatment using BiTE® antibodies such as blinatumomab (Nagorsen, D. et al., Leukemia & Lymphoma 50(6): 886-891, 2009) and solitomab (Amann et al., Journal of Immunotherapy 32(5): 452-464, 2009) are being clinically evaluated.

In some embodiments, the second therapy will comprise administration of a PARP inhibitor. Poly(ADP-ribose) polymerases (PARPs) are a family of enzymes involved in various activities in response to DNA damage. PARP-1 is a key DNA repair enzyme that mediates single strand break (SSB) repair through the base excision repair (BER) pathway. PARP inhibitors have been demonstrated to selectively kill tumor cells that harbor BRCA1 and BRCA2 mutations. In addition, pre-clinical and preliminary clinical data suggest that PARP inhibitors are selectively cytotoxic for tumors with homologous recombination repair deficiency caused by dysfunction of genes other than BRCA1 or BRCA2. In some embodiments, the PARP inhibitor is selected from the group consisting of ABT-767, AZD 2461, BGB-290, BGP 15, CEP 9722, E7016, E7449, fluzoparib, INO1001, JPI 289, MP 124, niraparib, olaparib, ON02231, rucaparib, SC 101914, talazoparib, veliparib, WW 46, or salts or derivatives thereof. In some embodiments, the anti-PARP therapy is administered at a dose equivalent to about 100 mg, about 200 mg, or about 300 mg of niraparib or a salt or derivative thereof. In some embodiments, the anti-PARP therapy is administered at a dose equivalent to about 100 mg of niraparib or a salt or derivative thereof. In some embodiments, the anti-PARP therapy is administered at a dose equivalent to about 200 mg of niraparib or a salt or derivative thereof. In certain embodiments, the anti-PARP therapy is administered at a dose equivalent to about 300 mg of niraparib or a salt or derivative thereof.

In some embodiments, the second therapy will comprise administration of a therapeutic cancer vaccine. Therapeutic cancer vaccines are designed to be used in people who already have cancer-they work against cancer cells that contain substances, called tumor-associated antigens, that are not present in normal cells or, if present, are at lower levels. Treatment vaccines can help the immune system learn to recognize and react to these antigens and destroy cancer cells that contain them. There are currently 2 FDA-approved vaccines that prevent cancer: a human papillomavirus (HPV) to prevent cervical, vaginal and vulvar cancers; and Hepatitis B vaccine to prevent liver cancer. The first FDA-approved oncolytic virus therapy is talimogene laherparepvec (T-VEC, or Imlygic®) based on herpes simplex virus type 1. In various embodiments, the vaccine therapy is selected from, but not limited to, treatment using therapeutic vaccines such as sipuleucel-T; treatment using dendritic cell vaccines, treatment using oncolytic virus therapy; and treatment using tumor antigen peptide vaccines.

Depending on the nature of the combinatory therapy, administration of the polypeptide therapeutic agents of the invention may be continued while the other therapy is being administered and/or thereafter. The polypeptide therapeutic agents may be administered prior to, concurrently with, or following the additional anti-cancer therapy, usually within at least about 1 week, at least about 5 days, at least about 3 days, at least about 1 day. The polypeptide therapeutic agents may be delivered in a single dose, or may be fractionated into multiple doses, e.g. delivered over a period of time, including daily, bidaily, semi-weekly, weekly, etc. The effective dose will vary with the route of administration, the specific agent, the dose of anti-cancer agent, and the like, and may be determined empirically by one of skill in the art.

In some embodiments, the treatment regime entails administration once per every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In still some embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically effective dose. In both prophylactic and therapeutic regimes, agents are usually administered in several dosages until a sufficient response has been achieved. Typically, the response is monitored, and repeated dosages are given if there is a recurrence of the cancer.

According to the present invention, compositions for the treatment of primary or metastatic cancer can be administered by parenteral, topical, intravenous, intratumoral, oral, subcutaneous, intraarterial, intracranial, intraperitoneal, intranasal, or intramuscular means. The most typical route of administration is intravenous or intratumoral although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. In some embodiments, the composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein. Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration. Preferably, a therapeutically effective dose of the polypeptide compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. The kit can further contain a least one additional reagent, for example a cytoreductive drug. The compositions may be provided in a unit dose formulation. Kits typically include a label indicating the intended use of the contents of the kit. The term label includes any writing, or recorded material supplied on or with the kit, or which otherwise accompanies the kit.

### Example 1

Pembrolizumab is an existing therapy for patients with locally advanced or metastatic urothelial carcinoma who had failed platinum-treated metastatic urothelial carcinoma. This was based on phase 3 trial in which patients were randomized to pembrolizumab (270) or chemotherapy (paclitaxel, docetaxel or vinflunine) (272). Pembrolizumab vs chemotherapy had a median OS of 10.3 months (95% Cl 8-11.8) vs 7.4 months, PFS of 2.1 months (95% Cl 2.0-2.2) vs 3.1 months, ORR of 21.1% (95% Cl 16.4-26.5) vs 11.4%. Complete remission with Pembrolizumab was 7%. Pembrolizumab was granted regular approval by the FDA.

Immune checkpoint inhibitor targeting agents like Pembrolizumab are effective in patients with tumors that express inflammatory signatures and contain resident immune cells commonly referred to as “hot tumors.” Tumors with a few or no immune cells (cold tumors) are unlikely to respond. Novel agents that recruit immune cells are, therefore, likely to improve patient response upon the current state of immunotherapy. Immune cell recruitment is in part limited by the tumor vascular barrier. EphrinB2 and its high affinity cognate receptor—EphB4—are transmembrane proteins that are induced in tumor vessels and regulate immune cell trafficking. Soluble extracellular fragment of EphB4 fused to albumin (sEphB4-HSA) blocks interaction between endogenously expressed EphrinB2 and EphB4 and blocks bidirectional signaling. sEphB4-HSA promotes immune cell traffic.

In this example, eight patients having naive locally advanced or metastatic urothelial cancer of the bladder were treated with sEphB4-HSA in combination with an anti-PD-1 antibody. The treatment regimen consisted of sEphB4-HSA 10 mg/kg IV infusion once a week plus Pembrolizumab (KEYTRUDA®) 200 mg IV infusion every 3 weeks. Tumor response was measured every 6 weeks. Baseline tissue or archival tissues are collected for biomarkers (in particular, PD-L1 IHC 22C3 PharmDx, a companion marker of Pembrolizumab/KEYTRUDA®).

Patient eligibility required that the patient had no prior immune therapy, were not eligible for platinum chemotherapy, or had previously declined or refused to receive chemotherapy. Ages ranged from 53 to 90. Six of the eight patients were men. Two had prior radical cystectomy (bladder resection) for bladder/urothelial cancer. Two patients had disease in the upper tract. Bellmunt 1 risk factor in 8 patients and bellmunt 2 risk factor in 1 patient. Hemoglobin less than 10 in 1 case. Seven patients were ineligible for platinum chemotherapy. 1 patient refused to receive chemotherapy. Pathology: variant (micropapillary in 1, squamous and sarcomatoid variants in 1 each. All patients had lymph node involvement, 7 had primary bladder cancer.

Results: Combination of soluble EphB4-albumin fusion and PD-1 antibody is well tolerated. As relates to biomarkers: 3 patients were PD-L1 positive (expression of 1% or greater) and all 8 patients were EphrinB2 positive (expression of 1% or greater). 6 patients were evaluated for response via CT scan. CT scan was pending in 1 case, and 1 patient discontinued therapy early. Surprisingly, 5 of the 6 patients who had a CT scan on therapy have remission, and all remissions are complete. Overall response rate for intent to treat is 71.4 percent. Among the PD-L1 positive cases, 2 of 3 patients responded to therapy. Durability of response is significant, and no patient had progressed after a follow of up to 10 months from response. It thus appears that combination of soluble EphB4-albumin fusion and PD-1 antibody has high response rates at 71.4% and EphrinB2 expression as a biomarker is highly predictive of response using the combination therapy.

### Example 2

The present inventors have previously reported on a Phase II clinical trial of sEphB4-HSA in combination with Pembrolizumab. Eligibility criteria for patients were locally advanced or metastatic urothelial cancer, who had previously failed (relapsed or refractory or intolerant) cisplatin containing regimen for locally advanced or metastatic disease or patients who had relapsed within 12 months of cisplatin containing neo-adjuvant therapy. Exclusion criteria were patients who had received prior checkpoint inhibitor targeting therapy.

In this study, treatment regimen consisted of sEphB4-HSA 10 mg/kg IV infusion once a week plus Pembrolizumab (KEYTRUDA®) 200 mg IV infusion every 3 weeks. Tumor response was measured every 6 weeks. Baseline tissue or archival tissues are collected for biomarkers (in particular, PD-L1 IHC 22C3 PharmDx, a companion marker of Pembrolizumab). Independent evaluation of the response was assessed by a blinded radiologic review. PD-L1 staining was performed at a reference laboratory. All patients were eligible for toxicity assessment. Primary end point for study is OS, secondary end points are ORR and PFS. Analysis for high-risk subsets includes squamous cell variant, upper urinary tract disease, liver metastasis, hemoglobin<10 mg/dl, level, and performance status over 0. 69 patients were enrolled in the study.

69 patients were accrued. Median age was 67. 80% of the patients were male. For biomarker analysis, 4 cases were excluded for lack of tissue (2 cases no tissue available, 2 cases tissue did not contain tumor). 65 patients with available tissues, biomarkers (EphrinB2 and PD-L1) were analyzed. 50 patients were positive for either EphrinB2 or PD-L1. 15 patients were negative for both EphrinB2 and PD-L1. 45 patient tissues were EphrinB2 positive (32 patient tissues were EphrinB2 positive only), 18 patients were PD-L1 positive (5 patients were PD-L1 positive only) and 13 patients expressed both EphrinB2 and PD-L1. 52 patients (35 EphrinB2 positive) had prior chemotherapy treatment.

It was determined that EphrinB2 positive patients have an overall response rate ORR) of 51%, compared to overall 37% for all patients. Importantly, both response rates are higher than the expected of 21% (based on historical data) if patients were treated with pembrolizumab alone. Four patients with metastatic urothelial cancer were treated with sEphB4-HSA alone during phase I portion of the study, and none had objective response. The activity of combination of sEphB4-HSA combined with PD-1 antibody thus appears to occur by the complementary functions where sEphB4-HSA promotes migration of T cells into the tumor while PD-1 antibody activates newly recruited and resident immune cells to achieve durable response.

In this study, sEphB4-HSA and pembrolizumab demonstrated a true synergistic activity, as the overall survival is 21 months, which is double that of 10 months observed with monotherapy using pembrolizumab, and 69% of the secondary responders remain in remission after 2 years.

### Example 3

Patients with relapsed and refractory Head and Neck Squamous cell carcinoma after chemoradiation have low response rates with cetuximab, systemic chemotherapy or check point inhibitor targeted therapy. HPV-negative HN SCC patients have poor outcome relative to HPV positive patients, including response to check point therapy. 2-year overall survival of HPV-associated head and neck cancer patients is 95% while 2-year overall survival of non-HPV-associated HNSCC is 62 percent. Thus, there is a large unmet need for novel therapies.

This clinical trial was a phase IIa, single arm, non-randomized, open-label trial of sEphB4-HSA combined with pembrolizumab (MK-7435) in patients with squamous cell carcinoma (SCC) of the head and neck. Patients of age 18 years old with Head and Neck SCC of the head and neck who met other eligibility criteria for study enrollment. Patients had locally advanced or metastatic disease that had progressed after 2 or more prior therapy, such as radio-chemotherapy. Patients must have been treated with systemic chemotherapy for first relapse, have adequate organ function, have measurable disease based on RECIST 1.1 (Immune-Related Response Evaluation Criteria in Solid Tumors), ECOG (Eastern Cooperative Oncology Group) performance status of 0 or 1, and had baseline tumor tissue available for analysis. The end points were toxicity, overall response rate (ORR), progression free survival (PFS) and overall survival (OS). HPV status and EphrinB2 biomarker expression were evaluated for outcome.

Planned enrollment was twenty-five patients. Treatment regimen was sEphB4-HSA 10 mg/kg once per week on days 1, 8 and 15, and Pembrolizumab 200 mg was given by intravenous infusion, once every 3 weeks, day 1 of each 3 week cycle. Therapy was given for a maximum of 24 months. Treatment was discontinued for confirmed radiographic disease progression, unacceptable adverse experiences, intercurrent illness, noncompliance with treatment, and patient or Tumor imaging was performed every 6 weeks (every 2 cycles) by computed tomography (CT) scan of the chest, abdomen and pelvis, plus a neck soft tissue CT scan. investigator decision to withdraw from the study. Tumor response was assessed according to RECIST. Patients who attained a confirmed complete response (CR) that had been treated for ≥24 weeks with pembrolizumab and had at least two treatments with pembrolizumab beyond the date of initial CR had the option of discontinuing treatment. Such patients, if experienced radiographic progression were eligible for up to one year of additional treatment, at the discretion of the investigator, if no cancer treatment was administered since the last dose of pembrolizumab.

Immunohistochemistry (IHC) was performed on patient tumor samples obtained at baseline and week 8 (cycle 2) on therapy. Biomarkers analyzed included EphrinB2, PD-L1, immune cell markers CD3, and CD8 for both collections. Patient tissue samples at baseline were sent to Caris Life Sciences for comprehensive tumor sequencing and PD-L1 using Monoclonal Rabbit Anti-PD-L1 Clone 28-8. Scoring for PD-L1 on both baseline and on-treatment tissue samples included tumor and immune cell membrane PD-L1 staining. Patients were determined to be PD-L1 positive if their tissue sample at baseline demonstrated >1% combined positive score (CPS). The scoring procedures and staining protocol are described in the instructions of the commercial assay for squamous cell carcinoma of the head and neck (SCCHN). IHC for EphrinB2 and immune markers was performed at the CLIA approved core laboratory and analyzed by an independent pathologist (I.S). EphrinB2 assay used Rabbit Monoclonal Anti-Ephrin B2 antibody. Scoring and analysis for EphrinB2 positivity at baseline and on-treatment biopsy was based on tumor cell membrane staining for EphrinB2. Patients were determined to be EphrinB2 positive if their tissue sample at baseline demonstrated ≥1% TPS. Scoring for both EphrinB2 and PD-L1 was based on a scale of 0 to 100 percent. p16 staining was done in the CLIA certified clinical laboratory as a routine service. IHC of immune markers was performed to assess immune cell infiltration into the tumor. This included staining for CD3, and CD8.

Demographics of the study population are shown below in Table 1.

TABLE 1

| | All Accrued | | HPV-Negative | |
|---|---|---|---|---|
| | Patients | | Patients | |
| Characteristic | (N = 25) | | (N = 15) | |
| Age | | | | |
| Median (range) - yr. | 61 | (31-79) | 61 | (31-79) |
| ≥75 yr. - no. (%) | 3 | (12) | 2 | (13) |
| Male sex - no. (%) | 20 | (80) | 11 | (73) |
| Race - no. (%) | | | | |
| White | 14 | (56) | 6 | (40) |
| Asian | 5 | (20) | 4 | (26.5) |
| Black | 2 | (8) | 1 | (7) |
| Other | 4 | (16) | 4 | (26.5) |
| Smoking or tobacco use - no. (%) | | | | |
| Current or former | 12 | (48) | 8 | (53) |
| Never | 13 | (52) | 7 | (47) |
| ECOG performance-status | | | | |
| score - no. (%) | | | | |
| 0 | 12 | (48) | 8 | (53) |
| 1 | 13 | (52) | 7 | (47) |
| ≥2 | 0 | | 0 | |
| Site of primary tumor - no. (%) | | | | |
| Larynx | 2 | (8) | 2 | (13) |
| Oral cavity | 4 | (16) | 3 | (20) |
| Oropharynx | 11 | (44) | 3 | (20) |
| Hypopharynx | 1 | (4) | 1 | (7) |
| Paranasal sinuses and nasal cavity | 2 | (8) | 1 | (7) |
| Nasopharynx | 4 | (16) | 4 | (26) |
| Salivary glands | 1 | (4) | 1 | (7) |
| Sites of metastatic disease - no. (%) | | | | |
| Lung | 15 | (60) | 7 | (47) |
| Liver | 5 | (20) | 3 | (20) |
| Bone | 5 | (20) | 2 | (13) |
| Adrenal Gland | 3 | (12) | 1 | (7) |
| Node Only | 6 | (24) | 5 | (33) |
| No. of previous lines of systemic cancer | | | | |
| therapy - no. (%) | | | | |
| 0 | 2 | (8) | 1 | (7) |
| 1 | 17 | (68) | 11 | (73) |
| ≥2 | 6 | (24) | 3 | (20) |
| No. of previous lines of systemic cancer | | | | |
| therapy in responders - no. (%) | | | | |
| 0 | 1 | (4) | 1 | (7) |
| 1 | 5 | (20) | 4 | (27) |
| ≥2 | 0 | | 0 | |
| Context of previous systemic therapy | | | | |
| regimen - no. (%) | | | | |
| Adjuvant therapy | 10 | (40) | 6 | (40) |
| Neoadjuvant therapy | 3 | (12) | 2 | (13) |
| Primary disease | 5 | (20) | 3 | (20) |
| Metastatic disease | 5 | (20) | 3 | (20) |
| No previous systemic therapy | 2 | (8) | 1 | (7) |
| Previous receipt of cetuximab - no. (%) | 4 | (16) | 2 | (13) |

The efficacy data is presented in Table 2.

TABLE 2
Efficacy in All Patients and HPV-Negative Patients

| | | | HPV-Negative | |
|---|---|---|---|---|
| | All Patients | | Patients | |
| | (N = 25) | | (N = 15) | |
| Evaluable for | 22 | (88) | 14 | (93) |
| Response - no. (%) | | | | |
| Overall Response | | | | |
| Rate - no. (%) | | | | |
| All Patients (ORR) | 6 | (24) | 5 | (33) |
| Evaluable Patients (ORR) | 6 | (27) | 5 | (36) |
| Best Overall | | | | |
| Response* - no. (%) | | | | |
| CR | 4 | (16) | 4 | (27) |
| PR | 2 | (8) | 1 | (7) |
| SD | 12 | (48) | 6 | (40) |
| PD | 7 | (28) | 4 | (27) |
| Overall Survival | 12.4 | (3.4-14.4) | 12.4 | (2.3-14.1) |
| (Months) (95% CI) | | | | |
| Progression Free Survival | 3.4 | (2.1-6.0) | 3.4 | (1.2-6.4) |
| (Months) (95% CI) | | | | |

*Rates including non-evaluable patients.

The combination of sEphB4-HSA plus pembrolizumab resulted in overall response in 7 patients, six of whom were among HPV negative patient population. Most of the HPV negative patients have high EphrinB2 expression. Despite small study population, it is apparent that targeting EphrinB2 combined with PD-1 antibody (pembrolizumab) offer higher likelihood of response. Overall survival (OS) among HPV negative patients was substantially higher with combination therapy than expected with Pembrolizumab alone (12.4 months vs. 8 months, respectively).

About 60% of patients were HPV-negative by p16 biomarker analysis. When comparing efficacy endpoints within HPV-negative patients, combination therapy also compared favorably to pembrolizumab alone; the ORR among HPV-negative patients receiving sEphB4-HSA plus pembrolizumab was 40%, and the ORR among HPV-negative patients receiving pembrolizumab alone was 14 percent. This substantial difference in response rates suggests that patients with non-HPV associated HN SCC, who typically have a worse prognosis than those with HPV-associated HN SCC, may benefit significantly from the combination of sEphB4-HSA plus pembrolizumab. Given the effects of single-agent sEphB4-HSA on immune cell transmigration and activation in the tumor microenvironment, plus the demonstrated efficacy of pembrolizumab in facilitating immune-mediated tumor killing, it is a reasonable outcome that combining the two therapies would result in greater immune cell infiltration into the tumor and a more efficacious response in patients.

Seventeen (68%) of 25 patients were EphrinB2 positive, while 12 of 15 HPV negative cases were EphrinB2 positive. All responding patients were EphrinB2 positive. Twelve (48%) of 25 patients were PD-L1 positive with CPS of 1% or greater. Among 13 patients with PD-L1 less than 1%, three achieved response (23%). Further analysis conducted for ORR based on EphrinB2 and PD-L1 positivity revealed: 4 (44%) of 9 patients achieved response in the EphrinB2 positive and PD-L1 positive group, 4 (50%) of 8 patients with observed response in the EphrinB2 positive and PD-L1 negative group These data suggest that EphrinB2 expression offers superior predictive value for response. Consistent with these findings is the lack of response in both the EphrinB2 negative and PD-L1 positive (N=3) or PD-L1 negative N=5) groups.

PD-L1 of 1% or greater was less common in this study population (11 of 25) which is consistent with the 3105 patient meta-analysis showing positivity in 42%, but substantially lower than reported such as 85% in Keynote 012. ORR in PD-L1 of 1% or higher was similar to those with PD-L1 less than 1%, 3 of 11 and 3 of 13 respectively. Response in PD-L1 negative 13 patients (3 of 13 or 23%) is surprising since PD-L1 less than 1% does shows very low response rate (4 of 46 of 8% in keynote 048). Results from the current study either reflect an anomaly of small numbers of offers and opportunity to address and unmet need.

The findings from this study provide support for the safety and efficacy of the combination of sEphB4-HSA plus pembrolizumab. There were no grade 4 adverse events and no treatment related deaths, and toxicities experienced by patients were both manageable and did not require discontinuation of treatment, except in one case of cardiomyopathy which is a known toxicity of pembrolizumab. Combination therapy improved significantly on ORR vs. pembrolizumab alone, particularly in the HPV-negative subgroup. Finally, biomarker analysis revealed an apparent difference in objective response rate in patients with tumors expressing EphrinB2 vs. patients who were EphrinB2 negative; in contrast, there was no similarly observed difference in response rate between PD-L1 positive vs. PD-L1 negative patients. Further studies are warranted to evaluate the tumor-suppressing process behind EphB4-EphrinB2 inhibition in the context of PD-1/PD-L1 inhibition, particularly in patients who do not express PD-L1.

### Example 4

Patients with metastatic urothelial carcinoma have poor prognosis after failure of standard first-line chemotherapy. Immune check point programmed death 1-programmed death ligand 1 antibodies have low response rates and thus there exists a major unmet need.

Patients with metastatic urothelial carcinoma that recurred or progressed after platinum-based chemotherapy received soluble EphB4-human serum albumin (sEphB4-HSA) in combination with pembrolizumab. Patients who were age 18 years or older with carcinoma of the urinary tract (the renal pelvis, ureter, bladder, or urethra) histologically confirmed predominantly urothelial without or with variants including squamous cell carcinoma, poorly differentiated cancer met eligibility for enrollment. Eligible patients also had disease progression after platinum-based chemotherapy for advanced disease or had recurrence within 12 months of receiving platinum-based adjuvant or neoadjuvant therapy for localized muscle-invasive disease. Patients may have received one or more additional systemic chemotherapy regimens for advanced disease before study enrollment. The primary end points were tolerability and overall survival (OS). The secondary end points were progression-free survival (PFS), objective response rate (ORR), duration of response, and toxicity. The expression of sEphB4-HSA target EphrinB2 was correlated with outcomes.

Patients were assigned to one of two cohorts: Cohort A patients had received only one prior platinum-based chemotherapy and cohort B patients had received at least one additional systemic chemotherapy regimen, with planned enrollment of 36 and 24 patients, respectively. Treatment regimen consisted of pembrolizumab 200 mg intravenously once on day 1 and sEphB4-HSA at 10 mg/m2 intravenously once daily on days 1, 8, and 15 once every 3 weeks. Treatment was continued until RECIST-defined disease progression, development of unacceptable toxicity, withdrawal of consent by the patient, decision by the investigator to discontinue treatment, or the completion of 2 years of therapy.

The median age was 67 years, with a male to female ratio of 59 to 11. Sixty-four patients enrolled with only one prior line of therapy while six patients had more than one line of therapy. Sites of disease at baseline included lymph nodes in 45 (64%), lungs in 24 (34%), liver in 18 (26%), and bone in nine (13%). Thirty-nine patients (56%) had an ECOG performance status of 0 and 31 (44%) patients had ECOG of 1. Bellmunt risk group distribution was as follows: 27% with no risk factors, 37% with one risk factor, and 36% with two or more risk factors. Fourteen (20%) patients had upper tract disease as their primary site of disease. Forty-six patients were EphrinB2-positive.

The efficacy data is presented in Tables 3 and 4.

TABLE 3

| | All Patients Treated | | Patients with EphrinB2+ | | |
|---|---|---|---|---|---|
| | (N = 70) | | Tumors (N = 46) | | |
| | # Pts. (%) | 95% CI | # Pts. (%) | | 95% CI |
| CR + PR | 26 (37%) | (26%, 48%) | 24 | (52%) | (38%, 67%) |
| (Objective | | | | | |
| Response) | | | | | |
| CR | 11 (16%) | (8%, 24%) | 11 | (24%) | (12%, 36%) |
| PR | 15 (21%) | (12%, 31%) | 13 | (28%) | (15%, 41%) |
| SD | 20 (29%) | (18%, 39%) | 13 | (28%) | (15%, 41%) |
| CR + PR + SD | 46 (66%) | (55%, 77%) | 37 | (80%) | (69%, 92%) |
| (Disease Control) | | | | | |
| PD | 24 (34%) | (23%, 46%) | 9 | (20%) | (8%, 31%) |

| | | |
|---|---|---|
| Median DOR | NR (13.3, NR) | NR (11.9, NR) |
| (95% CI) | 26 responders | 24 responders |
| in months** | 18/26 continuing | 16/24 continuing |
| | in response | in response |

**Based on KM with patients who stopped responding having an ‘event’
DOR = duration of response;
NR = not reached;
CI = confidence interval

TABLE 4

| | | | Patients with EphrinB2+ | |
|---|---|---|---|---|
| | All Patients (N = 70) | | Tumors (N = 46) | |
| | Resp/N | | Resp/N | |
| Sub/group | (%) | (95% CL) | (%) | (95% CL) |
| All patients | 26/70 (37%) | (26%, 49%) | 24/46 (52%) | (37%, 67%) |
| Age group | | | | |
| <65 y | 9/27 (33%) | (14%, 52%) | 9/18 (50%) | (24%, 76%) |
| >=65 y | 17/43 (40%) | (24%, 55%) | 15/28 (54%) | (33%, 73%) |
| Sex | | | | |
| F | 5/11 (46%) | (10%, 81%) | 5/8 (63%) | (19%, 100%) |
| M | 21/59 (36%) | (23%, 48%) | 19/38 (50%) | (33%, 67%) |
| ECOG | | | | |
| 0 | 14/39 (36%) | (20%, 52%) | 13/23 (57%) | (35%, 78%) |
| 1 | 12/31 (39%) | (21%, 57%) | 11/23 (48%) | (26%, 70%) |
| Bellmunt group | | | | |
| 0 | 9/19 (47%) | (23%, 72%) | 8/13 (62%) | (31%, 92%) |
| 1 | 9/26 (35%) | (15%, 54%) | 8/16 (50%) | (23%, 78%) |
| 2+ | 8/25 (32%) | (12%, 52%) | 8/17 (47%) | (21%, 74%) |
| HGB <10 | | | | |
| No | 23/62 (37%) | (25%, 50%) | 21/42 (50%) | (34%, 66%) |
| Yes | 3/8 (38%) | (0%, 81%) | 3/4 (75%) | (0%, 100%) |
| Histologic variant | | | | |
| No | 15/43 (35%) | (20%, 50%) | 15/24 (63%) | (42%, 83%) |
| Yes | 11/27 (41%) | (21%, 61%) | 9/22 (41%) | (19%, 63%) |
| Liver metastases | | | | |
| No | 23/52 (44%) | (30%, 58%) | 21/36 (58%) | (41%, 75%) |
| Yes | 3/18 (17%) | (0%, 36%) | 3/10 (30%) | (0%, 65%) |
| Visceral metastases | | | | |
| No | 6/8 (75%) | (36%, 100%) | 5/6 (83%) | (41%, 100%) |
| Yes | 20/62 (32%) | (20%, 44%) | 19/40 (48%) | (31%, 64%) |
| Disease site | | | | |
| Bladder or | 20/56 (36%) | (23%, 49%) | 18/37 (49%) | (32%, 66%) |
| Urethra (Lower) | | | | |
| Renal Pelvis or | 6/14 (43%) | (13%, 73%) | 6/9 (67%) | (28%, 100%) |
| Ureter (Upper) | | | | |
| Met sites | | | | |
| 0-1 organs | 14/26 (54%) | (33%, 74%) | 13/21 (62%) | (39%, 85%) |
| 2-4 organs | 12/44 (27%) | (14%, 41%) | 11/25 (44%) | (23%, 65%) |
| Prior Tx time since | | | | |
| completion of last therapy | | | | |
| ≤3 months | 7/26 (27%) | (9%, 45%) | 7/15 (47%) | (18%, 75%) |
| >3 months | 19/44 (43%) | (28%, 58%) | 17/31 (55%) | (36%, 73%) |
| Prior chemo completed | | | | |
| Neo-adjuvant/First Line | 25/64 (39%) | (27%, 51%) | 23/43 (54%) | (38%, 69%) |
| Second or 3^{rd} line | 1/6 (17%) | (0%, 60%) | 1/3 (33%) | (0%, 100%) |
| Prior treatment | | | | |
| Cisplatin | 20/52 (39%) | (25%, 52%) | 18/35 (51%) | (34%, 69%) |
| Carboplatin | 6/18 (33%) | (9%, 58%) | 6/11 (55%) | (20%, 90%) |
| EphrinB2 | | | | |
| Negative/Unknown | 2/24 (8%) | (0%, 20%) | 0 | — |
| Positive | 24/46 (52%) | (37%, 67%) | 24/46 (52%) | (37%, 67%) |

The median follow up was 22.9 months (range, 1.3-54.7). The regimen had acceptable toxicity. In the intent-to-treat analysis (N5 70), the median OS was 14.6 months (95% Cl, 9.2 to 21.5). Twenty-six (37%) patients had an objective response (95% Cl, 26 to 48). The median PFS was 4.1 (95% Cl, 1.5 to 5.7) months. Forty-six (66%) patients expressed EphrinB2, and among them, the median OS was 21.5 months (95% Cl, 12.4 to not reached), the ORR was 52% (95% Cl, 37 to 67), including a complete response rate of 24% (11 of 46; 95% Cl, 12 to 36). The median PFS was 5.7 (95% Cl, 2.7 to 27.9) months. Response was maintained at 6, 12, and 24 months in 88%, 74%, and 69% of the patients, respectively.

Combination therapy exceeded the expectations across all end points in the intent-to-treat analysis. In the EphrinB2-positive subgroup (n 5 46, 66%), all end points showed improvement over the intent-to-treat population. The combination of sEphB4-HSA and pembrolizumab appears synergistic with improved OS and ORR compared with historical data for programmed death 1/programmed death ligand 1 monotherapy.

### Example 5

To determine whether EphrinB2 expression is a prognostic marker for urothelial carcinoma and a predictor of response to checkpoint inhibition monotherapy, a retrospective study of patients with diagnosis of metastatic urothelial carcinoma who were treated with PD1/PD-L1 antibody monotherapy analyzed the expression of EphrinB2 and response among these patients. Briefly, tissues specimens were collected and stained for EphrinB2 and consider >1% expression was considered as biomarker positive. The biomarker expression was correlated with the reported outcome of PD1/PD-L1 monotherapy. Patient inclusion required the following: 1) at least one pathology specimen obtained prior to PD1/PD-L1 antibody therapy must be available and at least 3 unstained slides are required for tissue analysis; 2) at the beginning of PD1/PD-L1 monotherapy, radiographically measurable disease must be present; 3) treatment outcome must be describable as radiographic progression of disease (PD) which includes death, stable disease, partial response, and complete response; and 4) ECOG status 0, or 1, or 2 at enrollment if available. It was reasoned that by measuring the expression of EphrinB2 in the tissues from these patients it would be possible to determine if EphrinB2 expression was simply a surrogate for response to checkpoint inhibition.

To conduct the study, investigators obtained IRB approval for a retrospective evaluation of tissues from patients who had locally advanced or mUC, had progression of disease after systemic chemotherapy with cisplatin or carboplatin and then were treated with anti PD1/PD-L1 therapy. Patients included in the study had to have tissue available for biomarker analysis and radiographic assessment of response to immunotherapy (protocol and IRB approval are included in the appendix). Demographic, patient-specific and disease-specific data were collected. Tumor tissue blocks were freshly cut and stained for EphrinB2 and PD-L1 and reviewed in blinded fashion by the pathologist with experience in assessment of EphrinB2 and PD-L1 status (Dr. Imran Siddiqi, Norris Cancer Hospital, Keck School of Medicine).

Among 41 patients identified for the study, twenty-eight (28) patients fulfilled the requirements for inclusion in the study. Demographics of the study population are shown below in Table 5.

TABLE 5

Retrospective.
N = 28 (100)

Age - Year

| | |
|---|---|
| Median | 68.5 |
| Range | 41-85 |

Sex

| | | |
|---|---|---|
| Male (%) | 22 | (78.6) |
| Female (%) | 6 | (21.4) |

Sites of Primary Disease

| | | |
|---|---|---|
| Bladder or Urethra (%) | 26 | (92.9) |
| Upper Tract (%) | 2 | (7.1) |

Squamous Differentiation

| | | |
|---|---|---|
| Yes (%) | 7 | (25.0) |
| No (%) | 21 | (75.0) |

ECOG Performance Status Score - no.

| | | |
|---|---|---|
| 0 (%) | 14 | (50.0) |
| 1 (%) | 6 | (21.4) |
| >1 (%) | 8 | (28.6) |

Bellmunt Risk Factor

| | | |
|---|---|---|
| 0 (%) | 9 | (32.1) |
| 1 (%) | 7 | (25.0) |
| 2 or more (%) | 12 | (42.9) |

Liver Mets at Baseline

| | | |
|---|---|---|
| No (%) | 24 | (85.7%) |
| Yes (%) | 4 | (14.3%) |

Visceral Disease at Baseline

| | | |
|---|---|---|
| No (%) | 13 | (46.4%) |
| Yes (%) | 15 | (53.6%) |

Number of Metastasis

| | | |
|---|---|---|
| 0 | 3 | (10.7%) |
| 1 | 12 | (42.9%) |
| 2 | 7 | (25.0%) |
| ≥3 | 6 | (21.4%) |

Hemoglobin Concentration

| | | |
|---|---|---|
| <10 g/dL | 9 | (32.1%) |
| or ≥10 g/dL | 19 | (67.9%) |

Time Since Completion or Discontinuation of Most Recent
Chemotherapy Prior to PD-L1 Inhibitor Therapy

| | | |
|---|---|---|
| <3 Months (%) | 13 | (46.4%) |
| ≥3 Months (%) | 15 | (53.6%) |

Prior Chemotherapy

| | | |
|---|---|---|
| Cisplatin-based (%) | 19 | (67.8%) |
| Carboplatin-based/other therapy (%) | 7 | (25%) |
| MVAC | 2 | (7.1%) |

Immunotherapy Regimen

| | | |
|---|---|---|
| Atezolizumab | 10 | (35.7%) |
| Avelumab | 1 | (3.6%) |
| Durvalumab | 1 | (3.6%) |
| Nivolumab | 0 | (0.0%) |
| Pembrolizumab | 16 | (57.1%) |

Sites of Disease

| | | |
|---|---|---|
| Adrenal | 2 | (7.1%) |
| Bone | 5 | (17.9%) |
| Liver | 5 | (17.9%) |
| Lungs | 12 | (42.9%) |
| Lymph Nodes | 19 | (67.9%) |

Among the 28 patients included in this study, five (5) were responders; two (2) with complete remission and three (3) with partial response. The overall response 5 of 28 (17.9%). Three (3) additional patients had a best response as stable disease based on the radiographic assessment. Most patients in this series were treated with pembrolizumab. These data are consistent with the literature and expectations based on many prospectively conducted clinical studies with PD-1 or PD-L1 antagonistic antibodies in this patient population. Meta-analysis of PD-1/PD-L1 antagonistic antibodies studied and approved by the FDA for relapsed/refractory urothelial carcinoma after systemic chemotherapy have a response rate of 18%. (Tafuri et al. Clin GU Cancer. 2020, 18, 351-360).

EphrinB2 was expressed in 18 of the 28 cases and PD-L1 over 1% in 8 patients. Among the five responders, EphrinB2 was expressed at moderate to high level in two (2/18=11%). One of these patients had PD-L1 positive over 1% and had multiple sites of disease (liver, lung, lymph node). A second EphrinB2 positive patient who had partial response had PD-L1 level less than 1% and had lung as the site of metastatic disease. Three of the five responders had no EphrinB2 expression (3/10=30% ORR). Two of these patients had complete remission. Two of the three patients expressed PD-L1. All three patients had disease localized to the lymph nodes only.

TABLE 6
Response and Correlation with Biomarker (EphrinB2 and PD-L1)

| | |
|---|---|
| Ephrin B2 (+) | Ephrin B2 (−) |
| N = 18 (64.3%) | N = 10 (35.7%) |

| | | | | |
|---|---|---|---|---|
| PD-L1 (+) | 1/5 | (20%) | 2/3 | (66.7%) |
| N = 8 (28.6%) | | | | |
| PD-L1 (−) | 1/13 | (7.7%) | 1/7 | (14.3%) |
| N = 20 (71.4%) | | | | |
| Overall | 2/18 | (11.1%) | 3/10 | (30%) |

EphrinB2 positive have response rate of 11%, whereas EphrinB2 negative have a response rate of 30%. Thus, there is a 3-fold difference and suggests that EphrinB2 expression is predictive of a lower response to PD-L1 monotherapy.

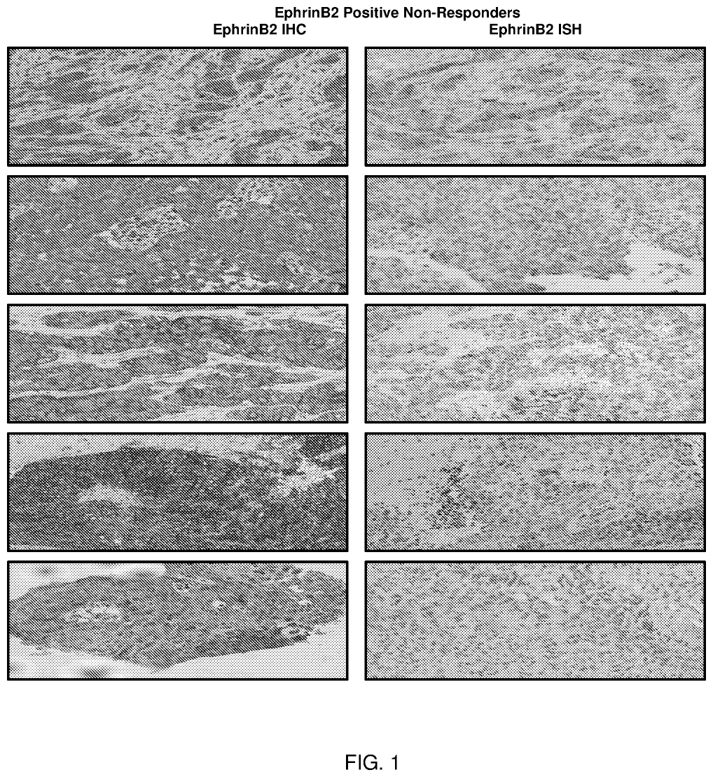

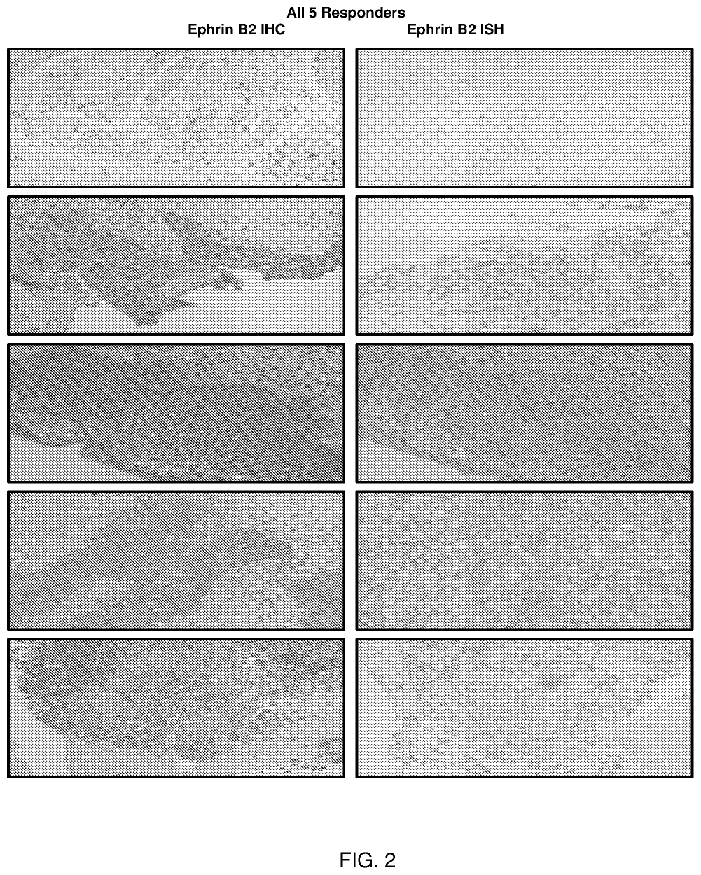

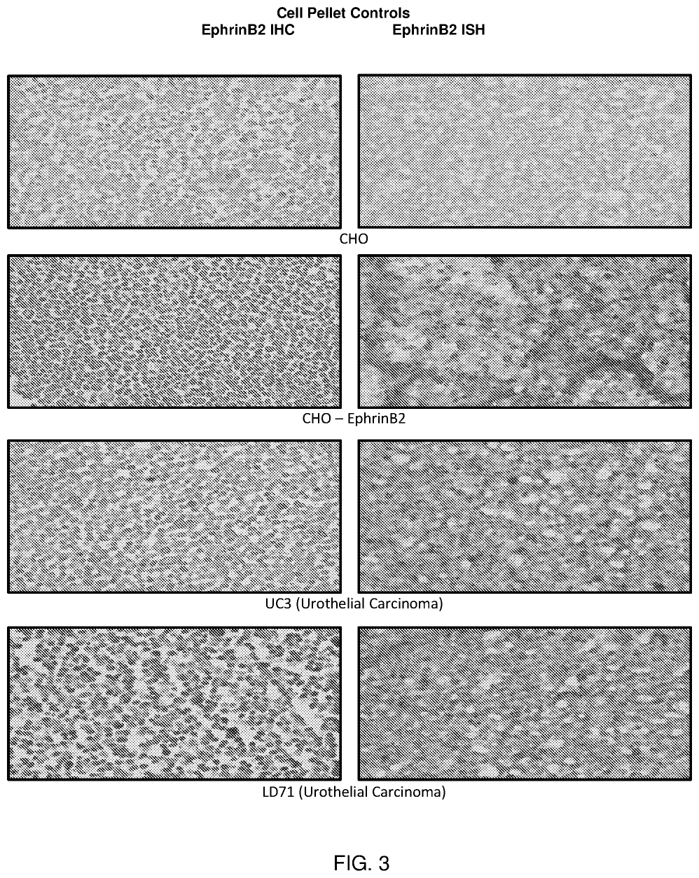

FIGS. 1-3 are pictures of EphrinB2 Immuno-histochemistry (IHC) and EphrinB2 In situ hybridization (ISH/RNAScope) of all 5 responders. Non-responders with high EphrinB2, and low EphrinB2 are also shown. Immunohistochemistry was performed with a recombinant monoclonal antibody generated in rabbit (Abcam 201512). The antibody showed specificity to EphrinB2 analyzed in isogenic CHO cell lines: wild type or ectopically expressing human EphrinB2 full length (CHO-WT, CHO-EphrinB2). It should be noted that EphrinB2 is a membrane protein thus requiring membrane localization in positive IHC staining. The absence of any staining or staining limited to signal in the nucleus or cytoplasm was considered negative (or nonspecific). EphrinB2 IHC positivity is defined by the membrane localization. Membrane localization in the presence of nucleus or cytoplasm staining is considered positive results. The present inventors have also validated the expression with in situ hybridization (RNAScope), technology developed by Advanced Cell Diagnostics (A.C.D). The assay has been confirmed in isogenic cell lines (CHO-WT as negative control, CHO-EphrinB2 as positive control, CHO-EphrinB1, as negative control demonstrating specificity even within its closely related protein).

A second retrospective study of patients (56) with diagnosis of metastatic urothelial carcinoma who were treated with PD1/PD-L1 antibody monotherapy analyzed the expression of EphrinB2 and response among these patients. Demographics of the study population are shown below in Table 7.

TABLE 7

| | Retrospective. | |
|---|---|---|
| | N = 56 (100) | |
| Age-Year | | |
| Median (%) | 73 | |
| Range (%) | 48-91 | |
| Sex | | |
| Male (%) | 37 | (66.1) |
| Female (%) | 19 | (33.9) |
| Sites of Primary Disease | | |
| Bladder or Urethra (%) | 33 | (58.9) |
| Upper Tract (%) | 23 | (41.1) |
| ECOG Performance Status | | |
| Score - no. | | |
| 0 (%) | 23 | (41.1) |
| 1 (%) | 22 | (39.3) |
| >1 (%) | 11 | (19.6) |
| Liver Mets at Baseline | | |
| No (%) | 38 | (67.9) |
| Yes (%) | 18 | (32.1) |
| Visceral Disease at Baseline | | |
| No (%) | 14 | (25.0) |
| Yes (%) | 42 | (75.0) |
| Number of Metastasis | | |
| 0 (%) | 0 | (0.0) |
| 1 (%) | 15 | (26.8) |
| 2 (%) | 16 | (28.6) |
| >2 (%) | 25 | (44.6) |
| Prior Chemotherapy | | |
| Cisplatin-based (%) | 29 | (51.8) |
| Carboplatin-based/other (%) | 7 | (12.5) |
| MVAC (%) | 0 | (0.0) |
| None (%) | 20 | (35.7) |
| Immunotherapy Regimen | | |
| Atezolizumab (%) | 6 | (10.7) |
| Avelumab (%) | 0 | (0.0) |
| Durvalumab (%) | 0 | (0.0) |
| Nivolumab (%) | 3 | (5.4) |
| Pembrolizumab (%) | 47 | (83.9) |
| Sites of Disease | | |
| Adrenal (%) | 3 | (5.4) |
| Bone (%) | 18 | (32.1) |
| Liver (%) | 18 | (32.1) |
| Lungs (%) | 20 | (35.7) |
| Lymph Nodes (%) | 36 | (64.3) |

TABLE 8
Response and Correlation with Biomarker (EphrinB2 and PD-L1)

| EphrinB2 | | | | | | |
|---|---|---|---|---|---|---|
| Expression | 0 | 1+ | 2+ | 3+ | 4+ | ORR |
| N = 56 (100) | 5/14 | 2/6 | 3/25 | 2/4 | 0/7 | 12/56 |
| | (35.7) | (33.3) | (12.0) | (50.0) | (0.0) | (21.4) |

| | |
|---|---|
| EphrinB2 Negative: | EphrinB2 Positive: |
| 7/20 (35.0) | 5/36 (13.9) |

EphrinB2 positive have response rate of 14%, whereas EphrinB2 negative have a response rate of 35%. And, importantly, patients with very high EphrinB2 (7 cases) had no response. This study thus validates the first study and collectively there were at least 12 patients with high EphrinB2 expression that had no response again demonstrating that EphrinB2 expression is predictive of a lower response to PD-L1 monotherapy.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the appended claims.

### SEQUENCE LISTINGS

The nucleic and amino acid sequences listed in the accompanying sequence listing are shown using standard letter abbreviations for nucleotide bases and three letter code for amino acids, as defined in 37 C.F.R. 1.822.

SEQ ID NO: 1 is the amino acid sequence of human ephrin type-B receptor precursor (NP_004435.3). Amino acid residues 1-15 encode a signal sequence.

(SEQ ID NO: 1)
MELRVLLCWASLAAALEETLLNTKLETADLKWVTFPQVDGQWEELSGLDEEQHSVRTYEV
CDVQRAPGQAHWLRTGWVPRRGAVHVYATLRFTMLECLSLPRAGRSCKETFTVFYYESD
ADTATALTPAWMENPYIKVDTVAAEHLTRKRPGAEATGKVNVKTLRLGPLSKAGFYLAFQD
QGACMALLSLHLFYKKCAQLTVNLTRFPETVPRELVVPVAGSCVVDAVPAPGPSPSLYCRE
DGQWAEQPVTGCSCAPGFEAAEGNTKCRACAQGTFKPLSGEGSCQPCPANSHSNTIGSA
VCQCRVGYFRARTDPRGAPCTTPPSAPRSVVSRLNGSSLHLEWSAPLESGGREDLTYALR
CRECRPGGSCAPCGGDLTFDPGPRDLVEPWVVVRGLRPDFTYTFEVTALNGVSSLATGP
VPFEPVNVTTDREVPPAVSDIRVTRSSPSSLSLAWAVPRAPSGAVLDYEVKYHEKGAEGPS
SVRFLKTSENRAELRGLKRGASYLVQVRARSEAGYGPFGQEHHSQTQLDESEGWREQLA
LIAGTAVVGVVLVLVVIVVAVLCLRKQSNGREAEYSDKHGQYLIGHGTKVYIDPFTYEDPNE
AVREFAKEIDVSYVKIEEVIGAGEFGEVCRGRLKAPGKKESCVAIKTLKGGYTERQRREFLS
EASIMGQFEHPNIIRLEGVVTNSMPVMILTEFMENGALDSFLRLNDGQFTVIQLVGMLRGIAS
GMRYLAEMSYVHRDLAARNILVNSNLVCKVSDFGLSRFLEENSSDPTYTSSLGGKIPIRWT
APEAIAFRKFTSASDAWSYGIVMWEVMSFGERPYWDMSNQDVINAIEQDYRLPPPPDCPT
SLHQLMLDCWQKDRNARPRFPQVVSALDKMIRNPASLKIVARENGGASHPLLDQRQPHYS
AFGSVGEWLRAIKMGRYEESFAAAGFGSFELVSQISAEDLLRIGVTLAGHQKKILASVQHMK
SQAKPGTPGGTGGPAPQY

SEQ ID NO: 2 is the amino acid sequence of human serum albumin preproprotein (NP_000468.1). Amino acid residues 25-609 encode the mature peptide.

(SEQ ID NO: 2)
MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

SEQ ID NO: 3 is the amino acid sequence of human ephrin type-B receptor-human serum albumin protein.

LEETLLNTKLETADLKWVTFPQVDGQWEELSGLDEEQHSVRTYEVCDVQRAPGQAHWLR
TGWVPRRGAVHVYATLRFTMLECLSLPRAGRSCKETFTVFYYESDADTATALTPAWMENP
YIKVDTVAAEHLTRKRPGAEATGKVNVKTLRLGPLSKAGFYLAFQDQGACMALLSLHLFYK
KCAQLTVNLTRFPETVPRELVVPVAGSCVVDAVPAPGPSPSLYCREDGQWAEQPVTGCS
CAPGFEAAEGNTKCRACAQGTFKPLSGEGSCQPCPANSHSNTIGSAVCQCRVGYFRART
DPRGAPCTTPPSADAHKSEVAHRFKDLGEENFKALVLIAFAQYLQQCPFEDHVKLVNEVTE
FAKTCVADESAENCDKSLHTLFGDKLCTVATLRETYGEMADCCAKQEPERNECFLQHKDD
NPNLPRLVRPEVDVMCTAFHDNEETFLKKYLYEIARRHPYFYAPELLFFAKRYKAAFTECCQ
AADKAACLLPKLDELRDEGKASSAKQRLKCASLQKFGERAFKAWAVARLSQRFPKAEFAE
VSKLVTDLTKVHTECCHGDLLECADDRADLAKYICENQDSISSKLKECCEKPLLEKSHCIAE
VENDEMPADLPSLAADFVESKDVCKNYAEAKDVFLGMFLYEYARRHPDYSVVLLLRLAKTY
ETTLEKCCAAADPHECYAKVFDEFKPLVEEPQNLIKQNCELFEQLGEYKFQNALLVRYTKK
VPQVSTPTLVEVSRNLGKVGSKCCKHPEAKRMPCAEDYLSVVLNQLCVLHEKTPVSDRVT
KCCTESLVNRRPCFSALEVDETYVPKEFNAETFTFHADICTLSEKERQIKKQTALVELVKHK
PKATKEQLKAVMDDFAAFVEKCCKADDKETCFAEEGKKLVAASQAALGL

SEQ ID NO: 4 is the amino acid sequence of human ephrin type-B receptor-human serum albumin protein.

LEETLLNTKLETADLKWVTFPQVDGQWEELSGLDEEQHSVRTYEVCDVQRAPGQAHWLR
TGWVPRRGAVHVYATLRFTMLECLSLPRAGRSCKETFTVFYYESDADTATALTPAWMENP
YIKVDTVAAEHLTRKRPGAEATGKVNVKTLRLGPLSKAGFYLAFQDQGACMALLSLHLFYK
KCAQLTVNLTRFPETVPRELVVPVAGSCVVDAVPAPGPSPSLYCREDGQWAEQPVTGCS
CAPGFEAAEGNTKCRACAQGTFKPLSGEGSCQPCPANSHSNTIGSAVCQCRVGYFRART
DPRGAPCTTPPSAPRSVVSRLNGSSLHLEWSAPLESGGREDLTYALRCRECRPGGSCAP
CGGDLTFDPGPRDLVEPWVVVRGLRPDFTYTFEVTALNGVSSLATGPVPFEPVNVTTDRE
VPPAVSDIRVTRSSPSSLSLAWAVPRAPSGAVLDYEVKYHEKGAEGPSSVRFLKTSENRAE
LRGLKRGASYLVQVRARSEAGYGPFGQEHHSQTDAHKSEVAHRFKDLGEENFKALVLIAF
AQYLQQCPFEDHVKLVNEVTEFAKTCVADESAENCDKSLHTLFGDKLCTVATLRETYGEM
ADCCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICENQD
SISSKLKECCEKPLLEKSHCIAEVENDEMPADLPSLAADFVESKDVCKNYAEAKDVFLGMFL
YEYARRHPDYSVVLLLRLAKTYETTLEKCCAAADPHECYAKVFDEFKPLVEEPQNLIKQNCE
LFEQLGEYKFQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNAETFTFHADICT
LSEKERQIKKQTALVELVKHKPKATKEQLKAVMDDFAAFVEKCCKADDKETCFAEEGKKLV
AASQAALGL

SEQ ID NO: 5 is the amino acid sequence of human ephrin type-B receptor-human serum albumin protein.

LEETLLNTKLETADLKWVTFPQVDGQWEELSGLDEEQHSVRTYEVCDVQRAPGQAHWLR
TGWVPRRGAVHVYATLRFTMLECLSLPRAGRSCKETFTVFYYESDADTATALTPAWMENP
YIKVDTVAAEHLTRKRPGAEATGKVNVKTLRLGPLSKAGFYLAFQDQGACMALLSLHLFYK
KCAQLTVNLTRFPETVPRELVVPVAGSCVVDAVPAPGPSPSLYCREDGQWAEQPVTGCS
CAPGFEAAEGNTKCRACAQGTFKPLSGEGSCQPCPANSHSNTIGSAVCQCRVGYFRART
DPRGAPCTTPPSAPRSVVSRLNGSSLHLEWSAPLESGGREDLTYALRCRECRPGGSCAP
CGGDLTFDPGPRDLVEPWVVVRGLRPDFTYTFEVTALNGVSSLATGPVPFEPVNVTTDRE
VPPAVSDIRVTRSSPSSLSLAWAVPRAPSGAVLDYEVKYHEKGAEGPSSVRFLKTSENRAE
LRGLKRGASYLVQVRARSEAGYGPFGQEHHSQTQLDESEGWREQDAHKSEVAHRFKDL
GEENFKALVLIAFAQYLQQCPFEDHVKLVNEVTEFAKTCVADESAENCDKSLHTLFGDKLC
TVATLRETYGEMADCCAKQEPERNECFLQHKDDNPNLPRLVRPEVDVMCTAFHDNEETFL
KKYLYEIARRHPYFYAPELLFFAKRYKAAFTECCQAADKAACLLPKLDELRDEGKASSAKQR
LKCASLQKFGERAFKAWAVARLSQRFPKAEFAEVSKLVTDLTKVHTECCHGDLLECADDR
ADLAKYICENQDSISSKLKECCEKPLLEKSHCIAEVENDEMPADLPSLAADFVESKDVCKNY
AEAKDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSALEVDETYVPKEF
NAETFTFHADICTLSEKERQIKKQTALVELVKHKPKATKEQLKAVMDDFAAFVEKCCKADDK
ETCFAEEGKKLVAASQAALGL

1-27. (canceled)

28. A method of diagnosing and selecting a subject with cancer for treatment using an EphB4-EphrinB2 inhibitor in combination with an immune stimulating drug as front-line therapy, the method comprising: i) detecting the level of EphrinB2 expression in a biological sample from a subject who has been diagnosed with a cancer; ii) selecting the subject for treatment using an EphB4-EphrinB2 inhibitor in combination with an immune stimulating drug when EphrinB2 expression is 1% or greater.

29. A method according to claim 28, wherein the cancer is refractory to standard anti-cancer therapies.

30. A method according to claim 28, wherein the subject has suffered relapse from prior treatment using standard anti-cancer therapies.

31. A method according to claim 28, wherein the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

32. A method according to claim 28, wherein the cancer is selected from the group consisting of a cancer that is a RAS (e.g., KRAS, HRAS, NRAS) mutant cancer, and a cancer with PTEN loss.

33. A method according to claim 28, wherein the EphB4-EphrinB2 inhibitor comprises a sequence selected from the group consisting of amino acids 1-197, 16-197, 29-197, 1-312, 16-312, 29-312, 1-321, 16-321, 29-321, 1-326, 16-326, 29-326, 1-412, 16-412, 29-412, 1-427, 16-427, 29-427, 1-429, 16-429, 29-429, 1-526, 16-526, 29-526, 1-537, 16-537 and 29-537 of SEQ ID NO: 1 (“sEphB4 polypeptide”) associated covalently or non-covalently with an albumin selected from the group consisting of a human serum albumin (HSA) (“sEphB4-HSA”) and bovine serum albumin (BSA) (“sEphB4-BSA”).

34. A method according to claim 31, wherein the sEphB4-HSA comprises the amino acid sequence selected from the group of sequences set forth in SEQ ID NO: 3, SEQ ID NO: 4, and SEQ ID NO:5.

35. A method according to claim 28, wherein the immune stimulating drug is an antagonist to an immune-checkpoint protein antigen selected from the group consisting of: SIRP, CD47, TIGIT, VISTA, CD152, CD279, CD274, LAG3, TIM3 and CD223.

36. A method according to claim 33, wherein the immune stimulating drug is selected from the group consisting of an anti-PD-1 Ab, anti-PD-L1 Ab, anti-CTLA Ab, anti-TIGIT Ab, anti-LAG3 antibody, and anti-TIM3 antibody.

37. A method according to claim 28, wherein the biological sample is selected from the group consisting of a tissue sample, a blood sample, a serum sample, a plasma sample, a cerebrospinal fluid (CSF) sample, an ascites fluid sample, and a cell culture sample.

38. A method of diagnosing and selecting a subject with cancer for treatment using an EphB4-EphrinB2 inhibitor as front-line therapy, the method comprising: i) detecting the level of EphrinB2 expression in a biological sample from a subject who has been diagnosed with a cancer; ii) selecting the subject for treatment using an EphB4-EphrinB2 inhibitor when EphrinB2 expression is 1% or greater.

39. A method according to claim 38, wherein the cancer is refractory to standard anti-cancer therapies.

40. A method according to claim 38, wherein the subject has suffered relapse from prior treatment using standard anti-cancer therapies.

41. A method according to claim 38, wherein the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

42. A method according to claim 38, wherein the cancer is selected from the group consisting of a cancer that is a RAS (e.g., KRAS, HRAS, NRAS) mutant cancer, and a cancer with PTEN loss.

43. A method according to claim 38, wherein the EphB4-EphrinB2 inhibitor comprises a sequence selected from the group consisting of amino acids 1-197, 16-197, 29-197, 1-312, 16-312, 29-312, 1-321, 16-321, 29-321, 1-326, 16-326, 29-326, 1-412, 16-412, 29-412, 1-427, 16-427, 29-427, 1-429, 16-429, 29-429, 1-526, 16-526, 29-526, 1-537, 16-537 and 29-537 of SEQ ID NO: 1 (“sEphB4 polypeptide”) associated covalently or non-covalently with an albumin selected from the group consisting of a human serum albumin (HSA) (“sEphB4-HSA”) and bovine serum albumin (BSA) (“sEphB4-BSA”).

44. A method according to claim 43, wherein the sEphB4-HSA comprises the amino acid sequence selected from the group of sequences set forth in SEQ ID NO: 3, SEQ ID NO: 4, and SEQ ID NO:5.

45. A method according to claim 38 further comprising a second therapy selected from the group consisting of: small molecule kinase inhibitor targeted therapy, surgery, cytoreductive therapy, cytotoxic chemotherapy, and immunotherapy; wherein the combination has a synergistic effect.

46. A method according to claim 38, wherein the biological sample is selected from the group consisting of a tissue sample, a blood sample, a serum sample, a plasma sample, a cerebrospinal fluid (CSF) sample, an ascites fluid sample, and a cell culture sample.

47. A method of diagnosing a subject with cancer comprising: i) detecting the level of EphrinB2 expression in a biological sample from a subject who has been diagnosed with a cancer and in a course of treatment with an immune stimulating drug; and ii) changing therapy when EphrinB2 expression is 1% or greater.
